(19) United States
(12) Patent Application Publication
(10) Pub. No.: US 2007/0122870 A1
(43) Pub. Date: May 31, 2007

(54) Method for water testing and devices and kit of components for use in such a method

(76) Inventors: Carol Mary Turley, Devon (GB); David Lowe, Devon (GB); Susana Barquero-Molina, Suffolk (GB)

Correspondence Address: NIXON & VANDERHYE, PC, 901 NORTH GLEBE ROAD, 11TH FLOOR, ARLINGTON, VA, 22203, US

(21) Appl. No.: 10/573,827

(22) PCT Filed: Sep. 30, 2004
(86) PCT No.: PCT/GB04/04189

§ 371 Date: May 17, 2006

(30) Foreign Application Priority Data
Sep. 30, 2003 (GB) 0322892.1
Jan. 9, 2004 (GB) 0400439.6

(51) Int. Cl. C12Q 1/04; C12M 1/34

(52) U.S. Cl. 435/34; 435/287.1

(57) ABSTRACT

A method for the determination of water quality, which method comprises: contacting a vessel comprising a population of bacteria with a water sample to be tested, said vessel comprising a semi-permeable material which allows the water sample to pass therethrough and contact said bacteria; and determining the growth rate of the bacteria and proportion of respiring bacteria in the vessel, thereby to determine the water quality of the water sample.

### FIELD OF THE INVENTION

The invention relates to a method for determining water quality and to devices and a kit of components for use in carrying out such a method.

### BACKGROUND TO THE INVENTION

Over the last two decades the enormous importance of bacteria in the marine environment has become apparent. For example, up to 95% of the Earth's biosphere is oceanic and the activity of bacteria sustains the productivity of the whole marine food chain and drives the biogeochemical cycles of the principal elements. Bacteria decompose organic matter into nutrients, which are reutilised by the phytoplankton and sustain oceanic production. They are also an important direct food source for many other organisms. Marine bacteria are ubiquitous and their abundance averages over a million cells per millilitre, forming the largest compartment of living biomass.

Human activity is adversely affecting the equilibrium of the biosphere and thereby the sustainability of the marine ecosystem. One means of interpreting this ecological impact in biological terms is to utilise bioassays. Bioassays generally include experimental techniques where selected organisms are exposed to a selected environmental matrix (water, sediment, etc) containing the chemical or mixture of chemicals of interest and where selected endpoints (for example growth) are studied.

Most of the bioassays currently used to test seawater quality are based on higher organisms, such as invertebrates and vertebrates. However, lower components of the food chain, such as bacteria, that sustain higher organisms receive little attention and yet their impact on the whole ecosystem could be considerable.

### SUMMARY OF THE INVENTION

We have adopted the novel approach of using caged bacteria as biomarkers so that it is possible to transplant selected cultures of marine bacteria into the field to test real-time and integrated toxic impact in the environment. Two bacterial endpoints have been chosen: growth as an indicator at the population level and as estimated, for example, by measuring in vivo optical density (OD) or turbidity (TRB); and respiration as an indicator at the sub-cellular level as measured, for example, by the fluorescence emitted by actively respiring bacteria, using the CTC redox dye.

According to the invention there is thus provided a method for the determination of water quality, which method comprises:

contacting a vessel comprising a population of bacteria with a water sample to be tested, said vessel comprising a semi-permeable material which allows the water sample to pass therethrough and contact said bacteria; and

determining the growth rate of the bacteria and proportion of respiring bacteria in the vessel,

thereby to determine the water quality of the water sample.

The invention also provides:

a detection device for use in a method of the invention which device is arranged to determine the optical density and/or turbidity of a bacterial culture and also to determine the level of fluorescence of a bacterial culture contacted with a redox dye;

a kit of components for use in carrying out a method of the invention, which kit of components comprises:

one or more vessels comprising a semi-permeable material which allows a water sample to pass therethrough;

a non-watertight carrying structure to carry the one or more vessels; and a member for securing the position of the one or more vessels within the carrying structure and/or for providing flotation of the carrying structure;

a deployment device suitable for carrying one or more vessels that each comprise a semi-permeable material which allows a water sample to pass into said vessels, the said deployment device comprising a non-watertight housing and means for securing the one or more vessels within the housing and/or means for providing flotation of the deployment device; and

a sampling device for use in a method of the invention, which sampling device comprises:

(i) a sampling chamber which is adapted to accept one or more vessels comprising a semi-permeable material which allows a water sample to pass therethrough; and

(ii) a closure member selectively switchable between two or more positions, wherein:

in one of the said positions the closure member seals the sampling chamber to prevent ingress of water into the sampling chamber when the device is submerged in water; and

in a second of the said positions the closure member is open to allow ingress of water into the sampling chamber when the device is submerged in water.

### BRIEF DESCRIPTION OF THE DRAWINGS

FIG. 1 shows a data sheet used during an ECOALERT toxicity assay.

FIG. 2 shows a scatter plot showing the changes in bacterial OD date from different toxic treatments during an ECOALERT toxicity assay. A similar plot can be prepared for CTC fluorescence.

FIG. 3 shows column plots calculated from FIG. 2 plot, showing the percentage of decrease of bacterial OD in each treatment, relative to non-toxic control values. A similar plot can be prepared for CTC fluorescence.

FIG. 4 shows a design for the ECOALERT deployment device: (a) basket; (b) floating rack; and (c) device assembled with dialysis tubes hanging secured inside, deployed in the water.

FIG. 5 shows a design for the ECOALERT deployment device: the main body is cut from a PVC pipe and the mesh is a coarse plastic screen. The different parts are held together by stainless steel screws and safety clips.

A. Upper part of the chamber, it has circular windows to allow water to flow through. It is open through the top and the bottom and it adjusts over the vessel rack of the lower part B. B. Lower part of the chamber where the vessels containing the bacteria are secured. It is open through the bottom to allow water to flow through. It is held to the upper part with stainless steel clips. C. Narrow PVC tube where a pole or cable can be inserted to deploy and/or anchor the chamber. D. Coarse mesh covers all the openings of the chamber to prevent debris and floating objects entering the chamber. E. Small buoy tied to the chamber for flotation.

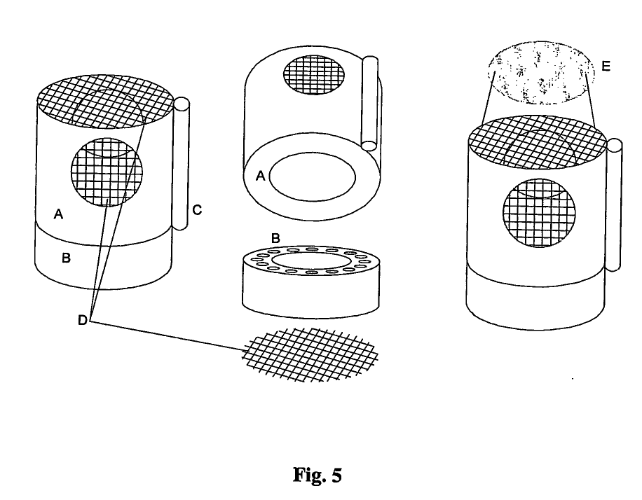

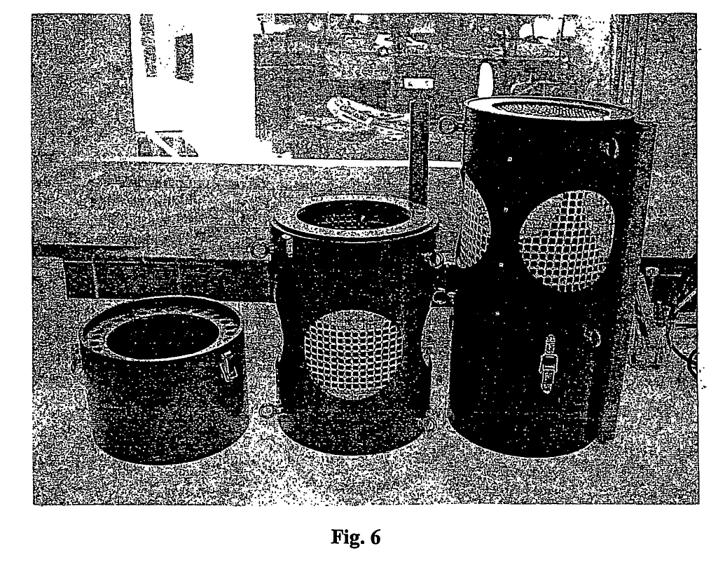

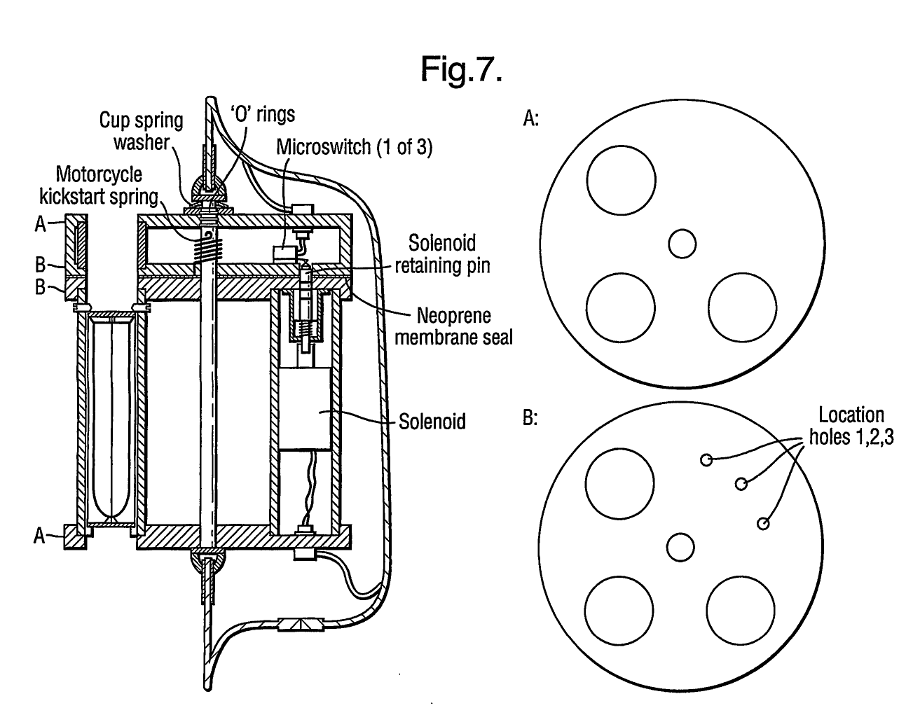

FIG. 6 shows a photograph of the design for the ECOALERT field application set out in FIG. 5.

FIG. 7 shows a sampling device useful in field deployment of ECOALERT.

### DETAILED DESCRIPTION OF THE INVENTION

The invention relates to a method for determining water quality. In particular, the method is suitable for determining whether a water sample contains pollutants, i.e. for determining the toxicity/contamination of the water sample.

The method, referred to as ECOALERT, is based on culturing a caged bacterial population in the presence of a water sample to be tested. The bacterial population is caged in a vessel which comprises a semi-permeable material. Thus, the bacteria cannot escape from the vessels, whereas the water sample and pollutants in that water sample may cross over the semi-permeable material into the vessel and thereby contact the bacteria.

The effect of the water sample on the bacterial population is assessed by determining changes in bacterial growth and respiration activity. These changes may be compared with changes that occur in relation to the same parameters when a caged bacterial population is contacted with a clean water control.

Growth may be measured by, for example, determining the optical density and/or the turbidity of the caged bacterial population and respiration activity may be measured by determining, for example, the fluorescence of the caged bacterial population after the bacteria have been stained with a redox dye.

The invention thus relates to a method for the determination of water quality, which method comprises: contacting a vessel comprising a population of bacteria with a water sample to be tested, said vessel comprising a semi-permeable material which allows the water sample to pass therethrough and contact said bacterial population; and determining the growth rate of the bacteria and proportion of respiring bacteria in the vessel, thereby to determine the water quality of the water sample.

ECOALERT can be run as a laboratory test of a water sample or deployed in the field for in situ analysis of water. ECOALERT has the potential for in situ field deployment in any aqueous environment, including open ocean and deep-sea In such conditions, data may be transferred by telemetry (i.e. via satellite).

The water sample to be tested may be any water sample where contamination is suspected. Thus, the water sample may be a marine, estuarine, coastal, freshwater (such as a lake, well or reservoir), or brackish sample, for example. The ECOALERT bioassay may be used to determine water quality around industrial activity, such as power stations, sewage discharges, fish farming, oil and gas industry process waters, drillings or other activities. That is to say, water samples to be tested may be obtained from such aqueous environments. Preferably, the water sample to be tested has not been sterilized, i.e. preferably the water sample is not sterilized prior to use in the ECOALERT bioassay.

If the ECOALERT assay is to be deployed in situ in the field, the water sample to be tested is, in this context, the aqueous environment in which the assay is deployed. ECOALERT may be deployed in, for example, marine, estuarine, coastal or freshwater (such as a lake, well or reservoir) environments. The ECOALERT assay may be deployed in aqueous environments which surround industrial activity, such as power stations, sewage discharges, fish farming, oil and gas industry process waters, drillings or other activities.

ECOALERT may be used to indicate the presence of a variety of different pollutants, for example heavy metals such as Cu, Hg, Cr, Pb, Zn, Cd or Ni, pesticides such as PCP or lindane, insect repellents such as DBP, detergents such as LAS, disinfectants such as Dettol, brominated flame retardants such as TBBA, pharmaceuticals such as bezafibrate, erythromycin, carbamazepine, clotrimazole, ibuprofen, miconazole, penicillin, salicyclic acid, tetracycline or paracetamol, crude oil, hydrocarbons such as PAHs, or mixtures of such pollutants.

The vessels used in ECOALERT comprise a semi-permeable material to allow the water sample with which they are contacted to pass over that semi-permeable material and into the vessel. All or part of a suitable vessel may comprise a semi-permeable material. For example, the vessel may comprise a panel or panels made of a semi-permeable material.

A vessel suitable for use in the invention may be of any shape, although the vessel will typically be tubular in form.

The vessels may be of any convenient volume, for example from about 1 ml to about 50 ml volume, in particular from about 5 ml to about 10 ml in volume.

A vessel suitable for use in ECOALERT will typically also comprise a closure means, for example a screw-cap or a snap-shut lid. The purpose of such closure means is so that the bacterial population may conveniently be loaded and secured within the vessel.

The semi-permeable material may be any semi-permeable material, but is typically dialysis tubing. Thus, the vessels used in ECOALERT are preferably dialysis tubes. Suitable commercially available dialysis tubes suitable for use in ECOALERT include Spectra/Por® Float-A-Lyzer® and Spectra/Por® Dispodialyzer® (both from SPECTRUM).

The ECOALERT assay may be carried out so that the effect of specific pollutants may be screened. This may be achieved by using a vessel comprising a semi-permeable material having a specific molecular weight cut off (MWCO). This will allow a vessel to discriminate for pollutants of particular size.

The molecular weight cut off of the semi-permeable material dialysis tubing may thus conveniently be from about 100 Da to about 300 kDa (MWCO), for example from about 20 kDa to about 100 kDa, in particular, about 25 kDa, about 50 kDa or about 60 kDa.

Vessels each comprising a semi-permeable material of a different MWCO may be used in the same assay to determine water quality of the sample for different types of pollutant.

A number of ECOALERT assays can be run simultaneously, i.e. a number of vessels can be contacted with the water sample to be tested at the same time. Indeed, it is recommended that each assay and each time point be carried out in triplicate. Thus, for an assay comprising 4 different treatments, 3 time points, 3 triplicates and 2 “blanks” will require (4×3×3)+(4×2)=44 dialysis tubes.

The vessels used in an ECOALERT assay can be the same or different. If they are different, vessels having walls comprised of a semi-permeable material having different MWCOs can be used. Thus, different contaminants may be screened for simultaneously.

A vessel for use in ECOALERT comprises a bacterium; more typically bacteria, for example a population of bacteria. The one or more bacteria are provided in the vessel in an aqueous environment. When more than one bacterium is used, the bacteria in the population may be from more than one genus. Thus, a vessel used in ECOALERT may comprise, for example, bacteria from two, three, four, five, up to 10 or even up to 50 genii. Moreover, each genus may be represented by bacteria from more than one species, for example two, three, four, five, up to 10 or even up to 50 species. Even if the vessel contains bacteria from only one genus, it may still comprise bacteria from more than one species. Typically, however, a vessel will comprise a population of bacteria from only one species.

The bacteria selected for use in ECOALERT will depend on the water sample to be tested. Suitable bacteria may be selected on the basis that they are capable of continued growth in the type of water sample to be tested. Typically, therefore, marine bacteria, estuarine bacteria, coastal bacteria, freshwater bacteria or brackish water bacteria will be preferred. Mixtures of different types of bacteria may be used.

In addition, suitable bacteria should be capable of growth at temperatures and salinities that may be encountered when field deployment of ECOALERT is used. For example, if natural seawater is to be tested, the bacteria selected will have to be capable of continued growth in natural waters of potentially diverse marine and/or estuarine environments, for example at temperatures of from about 5, 6 or 7° C. to about 20, 21 or 22° C. and at a salinity of from about 10.0% to about 35.5% salinity.

Typically, wild-type (unmodified) bacteria are used in ECOALERT. This is to ensure that the test represents the effects of toxicity on the natural microbial community. This information is important because the healthy function of bacteria is crucial for the sustainability of the whole marine environment. This should ensure greater flexibility for use of ECOALERT. However, mutant and/or genetically modified bacteria may be used in ECOALERT.

If seawater/estuarine water is to be tested, bacteria of the genus Pseudomonas or Vibrio are preferred. The species V. natrigens may preferably be used if bacteria of the genus Vibrio are to be used.

Alternatively, species from other different genii may be isolated from a particular environment and used with the ECOALERT assay for testing in that specific environment. In this event, preliminary tests may be necessary to identify the health hazard, growth requirements and tolerance to salinity and temperature range for the species to be used.

Generally, bacteria suitable for use in ECOALERT will be non-pathogenic. Preferably, any bacteria used in ECOALERT will belong to Hazard Group 1, defined by the Advisory Committee on Dangerous Pathogens (ACDP) as “a biological agent unlikely to cause human disease”.

Typically, a preculture of bacteria is prepared the day before the ECOALERT assay is initiated. This preculture is then used to inoculate the experimental culture for the ECOALERT test itself. The experimental culture is typically cultured for a period of time before the start of the test. The exposure time of ECOALERT runs from a typical minimum of 6 hours to a maximum of 24 h, 48 h or 72 h or even longer as required.

The preculture may be carried out according to methods well known to those skilled in the art. The media and culture conditions used may vary with the particular bacteria used in the ECOALERT test. Typically, however, a culture may be grown overnight by inoculating bacteria from an agar stock in about 10 ml to about 100 ml, such as 50 ml, of a suitable growth medium (for example 210 NCI Seawater Yeast Peptone growth medium) diluted 1:10 (for example with uncontaminated water of the type to be tested). Alternatively, a different dilution may be necessary if a different growth medium is to be used with bacterial strains other than Pseudomonas sp. NCIMB 1534) or Vibrio natriegens (NCIMB 857). Suitable dilutions will be apparent to those skilled in the art.

The preculture may then be used to inoculate a larger volume of growth medium, for example 500 ml (again typically diluted 1:10 with uncontaminated water of the type to be tested). The preculture is inoculated into an amount of growth media so that the optical density of the resulting solution approaches about 0.02 (OD taken at about 540 nm wavelength). The new subculture is then typically cultured under the same conditions as the preculture.

The larger subculture may be used to fill the vessels, for example dialysis tubes, once the bacteria enter the exponential growth phase. To that end, the optical density of the subculture is checked regularly, typically over 1 to 2 h.

Optimally, the assay should start when the bacteria enter the exponential phase, i.e. at an OD (taken at a wavelength of about 540 nm) of about 0.04. The number of vessels to be used in ECOALERT and the volume of those vessels determines the amount of the subculture that is required.

When the bacteria in the subculture have entered the exponential phase the ECOALERT assay may be initiated. The vessels may need to be emptied if they have been stored in liquid and pretreated according to any manufacturers recommendations.

For example, dialysis tubes are usually shipped in a preservative solution and will need to be rinsed thoroughly in distilled water prior to use. Typically, dialysis tubes may be subjected to UV for a few minutes and then stored over night in distilled water in the cold, for example at about 4° C. Dialysis tubes should not be left in the air to dry because the membrane rapidly deteriorates, shrinks and stiffens and becomes opaque, brittle and very difficult to handle. To avoid this, the tubes are refilled with the bacterial suspension immediately after they have been emptied.

Alternatively, in the case of field studies, it may be more convenient to prepare the subculture and fill the vessels before the field test day. In this case, the subculture OD can be adjusted between about 0.01 and about 0.04 (taken at a wavelength of about 540 nm). The vessels filled with the subculture as well as the “blank” vessels filled with medium only may be individually encapsulated inside suitable sterile closed containers also filled with medium only. The vessels in their capsules are then stored in the cold (i.e. <5° C.) until the next day in, for example, an incubator or a fridge. They may then be transported in this fashion to the test site, where they are removed from their capsules and deployed. Alternatively, they can be activated for about ½ to about 1 hour at the in situ temperature inside the incubator prior to deployment.

To initiate the ECOALERT assay, a vessel may be filled with the subculture. A “blank” vessel may be filled with medium only, i.e. minus bacteria The vessel (and the “blank” vessel if used) may then be contacted with the water sample to be tested. Generally, this will involve simply placing the vessel in the water sample to be tested. A vessel may be submerged entirely in the water sample to be tested or only partially submerged within the water sample to be tested. Of course, at least some of the semi-permeable material of the vessel must be below the surface of the water sample to be tested so that the water sample may cross the semi-permeable material.

The vessel may conveniently be placed in a basket adapted to accept the vessel. The basket may contain a flotation member so that the vessel floats on the surface of the water sample to be tested. The flotation member may thus provide flotation of the basket containing the vessel and may also secure the position of the vessel within the basket.

Replicate vessels may be deployed in a floating array. This may help to assess surface plumes or slicks for example. In this way, differences in surface pollution may be assessed. Alternatively, replicate vessels may be arranged at different depths within the water sample. This may be helpful in assessing a profile of depths. A basket may be used which accepts the vessels arranged such that, when the basket is deployed in a water sample, the vessels are at floated at different positions on the surface of the water and/or different depths within that water sample. The ECOALERT assay may be carried out by withdrawing a test sample from a vessel at a chosen time point or time points.

Thus, the invention provides a method for the determination of water quality, which method comprises:

contacting a vessel comprising a population of bacteria with a water sample to be tested, said vessel comprising a semi-permeable material which allows the water sample to pass therethrough and contact said bacteria;

withdrawing a test sample from the vessel; and

determining the growth rate of the bacteria and proportion of respiring bacteria in the test sample,

thereby to determine the water quality of the water sample.

A test sample may be withdrawn from a vessel at any predetermined time point after initiation of the assay. A test sample may thus be withdrawn 1, 2, 4, 6, 8, 12, 16, 24, 32, 48, 72 hours or even longer after the assay is begun, i.e. after the vessel is contacted with the water sample to be tested.

Usually, the vessel is mixed before a test sample is withdrawn. Any volume of test sample may be withdrawn from a vessel, for example from about 1 to about 10 ml, such as about 2 ml. If more than one vessel is being used in the assay, the entire contents of a vessel may be withdrawn for use as a test sample.

More than one sample may be withdrawn from a vessel, each test sample being withdrawn at different time points. For example two, three, four, five, six or more test samples may be withdrawn (each at different time points). More than one test sample may be withdrawn from a vessel at the same time point, i.e. replicate test samples may be withdrawn.

However, better results are obtained if each vessel is sampled only once. Sampling the contents of the same vessel repeatedly increases the risk of contamination. It also increases the total handling time because it is necessary to condition the tube after sampling to remove the air space created. This means that a sufficient number of tubes must be anticipated for each test so that an appropriate number of replicates and time-points can be processed.

The first test sample may be withdrawn as soon as possible after the ECOALERT bioassay has been initiated and preferably within the first half hour of beginning the bioassay.

In a preferred version of the ECOALERT bioassay, a first test sample is withdrawn within the first half hour of the start of the bioassay, a second test sample is withdrawn at about 6 hours (preferably from a different vessel) and a third test sample at about 24 hours (again, preferably from a vessel different than those from which the first two test samples were withdrawn).

In a further preferred version of the ECOALERT bioassay, a first sample is withdrawn within the first half hour of the start of the bioassay and then further samples are withdrawn at hourly intervals, up to at least 6 hours, for example 12 hours, 18 hours, 24 hours or even longer, such as up to 48 hours or 72 hours or even longer. Preferably each of the test samples is withdrawn from a different vessel.

Test samples may be withdrawn hourly for the duration of the ECOALERT bioassay, for example up to 24, 48 or 72 hours or even longer. Again, preferably each of the test samples is withdrawn from a different vessel.

The advantage of withdrawing a plurality of test samples in this way is that the effect of any pollutants in the water to be tested may be integrated over time.

In a further version of the ECOALERT assay, more than one test sample is withdrawn from the vessel(s) at the or each time point to be sampled, for example two, three or four test samples may be withdrawn at the or each time point to be sampled. If two test samples are withdrawn, one of the test samples may be used in the remainder of the ECOALERT assay and one of the test samples may be stored, for example at 4° C., for analysis at a later time.

The vessels or the test samples are processed so as to determine the growth rate of the bacteria in the test sample and the proportion of respiring bacteria in the test sample. This is done so as to determine the effect of any pollutants in the water to be tested at the molecular level (as given by the respiration activity) and at the population level (as given by growth). Bacterial cells double in number rapidly and several generations will have grown during the period of exposure to the water being tested.

The growth rate of the bacteria in a vessel or test sample is determined by measuring the optical density and/or the turbidity of the bacterial population in the vessel or test sample. This may be carried out using, for example, a spectrophotometer.

The vessel may itself be suitable for use directly in, for example, a spectrophotometer. Alternatively, a test sample may be transferred to an appropriate cuvette. The optical density and/or turbidity will generally be determined at a wavelength of from about 500 to about 600 nm, for example at a wavelength of about 515 nm or at about 540 nm.

The proportion of respiring bacteria in the vessel or test sample is determined by contacting the vessel or test sample with a fluorescent redox dye and measuring the resulting level of fluorescence. A suitable redox dye may be added to the vessel or test sample. A suitable redox dye will typically be water soluble.

Redox dyes are tetrazolium salts that are metabolically reduced by the active cells to form intracellular formazan products, which are coloured or fluorescent. Suitable redox dyes that may be used in the invention include 3-(4,5-dimethylthiazol-2-yl)-2,5-diphenyltetrazolium bromide (MTT), 5-(3-carboxymethoxyphenyl)-2-(4,5-dimethylthiazolyl)-3-(4-sulpho-phenyl)tetrazolium, inner salt (MTS), 2,3-bis(2-methoxy-4-nitro-5-sulphophenyl)-5-[(phenylamino)carbonyl]-2H-tetrazolium hydroxide, sodium salt (XTT) and 5-cyano-2,3-ditolyl tetrazolium chloride (CTC).

The resulting level of fluorescence is typically determined using a fluorometer. The vessel may be suitable for use directly in a fluorometer or, alternatively, the test sample may be transferred into a container which is suitable for use in a fluorometer.

The ECOALERT method is typically carried out using a new fluorometric application of the classical 5-cyano-2,3-ditolyl tetrazolium chloride (CTC) method. CTC is a vital redox dye.

CTC was firstly exploited for medical research but latterly has been extensively used in environmental studies to determine the number of metabolically active bacteria, generally by using epifluorescence microscopy or flow cytometry. However, epifluorescence counting of bacteria is a time-consuming task. The use of flow cytometry accelerates the procedure, but then it is an expensive technique. The spectro-fluorometric technique developed for ECOALERT combines rapidity, simplicity and low cost.

Use of CTC in the ECOALERT method has been developed in cultures of marine bacteria but in theory can be applied to any suspension of living cells. In the case of marine bacteria, the ECOALERT assay is typically carried out with a minimum concentration of 10⁷ cells per ml.

For use in ECOALERT, a 50 mM solution of CTC is prepared freshly in water, for example ultra-pure Milli-Q water, (100 mg CTC in 6.6 ml) and generally filtered through a 0.2-μm syringe filter. The solution should be kept in the dark and refrigerated until use, typically it should be used in the day of preparation.

The CTC solution may be added to a vessel or to a harvested test sample. The CTC solution is added to achieve a final concentration of from about 11.0 mM to about 5.0 mM CTC and is typically. A preferred final concentration is 2.5 mM CTC (for example, a 1900 μl test sample is mixed with 100 μl of 50 mM CTC solution) because this minimises the potential toxic effects of CTC, but is still a saturating concentration

The vessel/test sample: CTC mixture may then be incubated in the dark at from about 10° C. to about 37° C., preferably at about 28° C., for from about 30 minutes to about 2 hours, for example 1 hour, typically without stirring. It is advisable to prepare parallel “blank” samples (minus bacteria) to account for non-biological reduction of CTC using cell-free samples. Thus, it is preferable that bacteria-free blanks are used with every set of replicates for all measurements, prepared from medium-only without cells.

After the incubation, the fluorescence emitted by the reduced CTC-formazan is measured, for example using a fluorometer. Typically, the level of fluorescence is determined by exciting the bacterial population in the vessel or test sample using light of a predetermined intensity at a first predetermined wavelength and measuring the intensity of fluorescence at a second predetermined wavelength.

The first predetermined wavelength (the excitation wavelength) is typically from about 450 nm to about 550 nm, for example about 488 nm. The second predetermined wavelength (the emission wavelength) is typically from about 600 to about 650 nm, for example about 630 nm.

The optical density (and/or turbidity) of the same vessels or test samples may also be measured using a spectrophotometer (for example at a wavelength of about 540 nm). The optical density is typically also measured before adding the CTC dye to the test sample.

To calibrate the measurements of CTC fluorescence and optical density against bacterial abundance, the vessels or samples of CTC-stained bacteria may be processed afterwards to prepare microscopic slides. Alternatively, cell counting can be carried out using flow cytometry. From each vessel or test sample, 1 ml of sample may be preserved with 10% glutaraldehyde and kept in the fridge overnight. On the next day, the preserved samples are counter-stained with 10% DAPI (4′6-diamidino-2-phenylindole, Sigma) for 5-10 minutes and filtered through black 0.2 μm membrane filters (Nuclepore®, Poretics). The filters are mounted onto microscopic slides using low fluorescence immersion oil and counted in an epifluorescence microscope. From each field the number of total (DAPI stained) and respiring (CTC stained) bacteria are recorded.

The raw data may be recorded as follows: optical density (A540 nm wavelength), before and after the incubation with CTC; CTC fluorescence (488 nm excitation and 630 nm emission wavelengths); total number of DAPI-stained cells viewed under epifluorescence with UV excitation (340-380 nm wavelength); and number of CTC-stained cells viewed under epifluorescence with blue excitation (450-490 nm wavelength)

To calculate the results, optical density may be plotted against total epifluorescence DAPI counts from the same vessels or samples. The resultant function can be used to convert optical density values to abundance of bacteria Likewise, CTC fluorescence may be plotted against epifluorescence CTC+ counts of the same test samples to derive the relationship that can be used to convert CTC fluorescence into number of actively respiring, CTC-reducing bacteria. These two relationships can then be used to estimate the proportion of actively respiring bacteria within the total population.

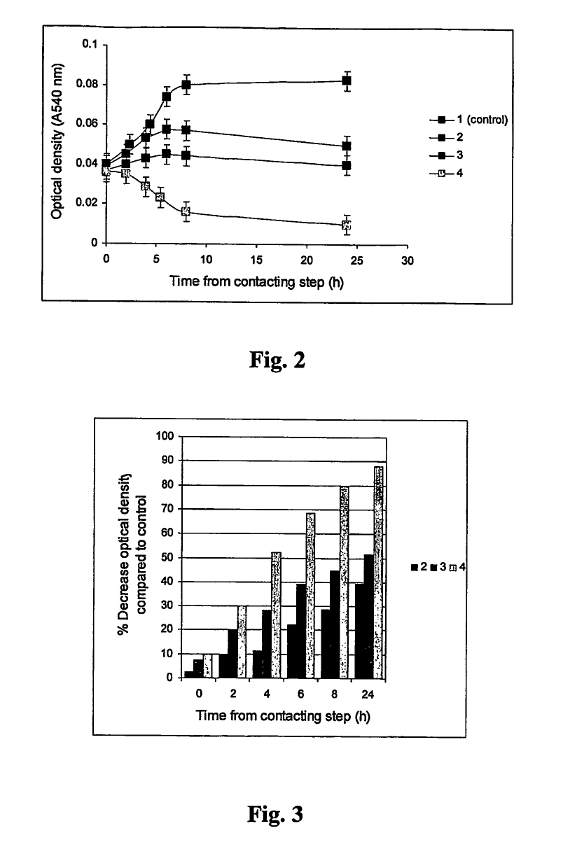

In general, the data obtained from the two types of analysis carried out on the vessels or test samples is processed to calculate the statistical average and standard deviation for each group of test samples at each treatment/time. The “blank” values are subtracted from the averages for both OD and CTC. OD blanks should be near zero and any higher value should be suspected of contamination. CTC blanks may vary, but should decrease with time. The results may be summarised in a scatter plot form to show changes in bacterial cultures with time (see FIGS. 2 and 3).

Typically, a contaminated vessel or test sample will show less bacterial growth and/or respiration in comparison with a non-contaminated vessel or test sample. That is to say, in the method of the invention, a decrease in bacterial growth rate and/or in the proportion of respiring bacteria in the vessel of test sample as compared to the corresponding bacterial growth rate and/or proportion of respiring bacteria in a control water sample is indicative of a decrease in water quality, i.e. indicative of toxicity/pollution/contamination. Thus, inhibition of bacterial growth and/or the proportion of respiring bacteria in the method of the invention is indicative of a decrease in water quality.

However, the ECOALERT bioassay can also detect positive effects in situations when a substance within the water sample to be tested stimulates or promotes growth and/or respiration of the bacteria in the vessel or test sample. Such substances may of course be undesitable toxins/pollutants.

Even if there is no clear effect observed in the redox dye indicator alone, there is an advantage to using simultaneously the bacterial indicators OD (or turbidity) and redox dye, for example CTC, fluorescence. It is possible to use the ratio of respiration/growth, which is an estimator of the proportion of respiring or active bacteria in the population, as an indirect indicator of the metabolic status of the population.

In some trials of ECOALERT, the study of the CTC/OD (or CTC/TRB) ratio gave some additional information. For example, in the case carbamazepine, an initial positive effect on the CTC/TRB ratio that comes down to control values after 24 hours was observed, although the trend suggests that there could be a negative effect in a longer exposure. In the case of PCP, at the end of the exposure to PCP the CTC/TRB ratio confirmed that PCP caused a loss of respiring cells in the culture compared to the control.

Typically, in particular during field tests, it is convenient to measure the OD (and/or turbidity) and CTC fluorescence using a device which is capable of making both measurements.

Accordingly, the invention provides a detection device which is arranged to determine the growth rate and proportion of respiring bacteria in a bacterial culture. The detection device is preferably arranged to determine the optical density and/or turbidity of a bacterial culture and also to determine the level of fluorescence of a bacterial culture contacted with a redox dye.

Thus, the detection device comprises means for determining the optical density and/or turbidity of a bacterial culture and means for determining the level of fluorescence of a bacterial culture mixed with a redox dye.

The detection device is typically suitable for use in the method of the invention.

The means for determining the optical density of a bacterial culture may comprise means determining the optical density at a wavelength of from about 500 to about 600 nm, for example at a wavelength of about 515 nm or at about 540 nm. The means for determining the optical density of a bacterial culture may comprise means for illuminating the bacterial culture, i.e. a light source, for example a green LED, and means for detecting emission of light from the bacterial culture at about 540 nm. The detecting means may be, for example, a photodiode detector.

The means for determining the level of fluorescence of a bacterial culture mixed with a redox dye may comprise means for exciting the bacterial culture. Suitable excitation means may excite the bacterial culture by the use of light, typically at a predetermined intensity, of a first predetermined wavelength. The means for determining the level of fluorescence of a bacterial culture mixed with a redox dye may also comprise means for measuring the intensity of fluorescence at a second predetermined wavelength.

Typically, the means for determining the level of fluorescence of a bacterial culture contacted with a redox dye comprises a light source for emitting said light of said first predetermined wavelength and a detector for detecting light of said second predetermined wavelength.

The first predetermined wavelength may be from about 450 nm to about 550 nm, for example about 488 nm. The second predetermined wavelength may be typically from about 600 to about 650 nm, for example about 630 nm.

The light source for emitting said light of said first predetermined wavelength may be a blue LED. The detector for detecting light of said second predetermined wavelength may be a photodiode.

The detection device will generally comprise a chamber which is adapted so as to accept a vessel (as described above) or some other container carrying the bacterial culture to be tested (for example a test sample as described above). The detection device may comprise more than one such chamber, for example two, three or four such chambers.

A vessel or container suitable for use in the device of the invention will be suitably transparent to the wavelengths of light which are used to excite the bacterial cultures and to the wavelengths of light at which optical density (and/or turbidity) and fluorescence are measured. Conveniently then, a suitable container may be formed from a plastics material such as polystyrene. The container may therefore be a polystyrene cuvette. The detection device will therefore generally comprise a chamber adapted to accept such a cuvette or cuvettes. The volume of a suitable container will typically of from about 1 ml to about 5 ml, such as 2 ml or 3 ml.

Typically, the device is a two-channel device having a switch which allows the user to toggle between the fluorescence and optical density (and/or turbidity) measurements. Generally, the device will be portable. That is to say, it should be suitably small and lightweight so that it may be hand-held. Preferably, it can easily fit into a shirt or jacket pocket. To this end, the device may be battery operated, for example using AA or AAA batteries.

The device may be adapted so that it floats, thus reducing the chance of the device being lost whilst carrying out ECOALERT in situ in the field. Ideally therefore, the detection device should be watertight.

The device may be adapted so that the data generated in the optical density and fluorescence measurements may be stored in the device. If the device is so adapted, the device will preferably be further adapted so that data can be downloaded onto a computer for subsequent analysis of the results.

A deployment device and a kit of components, typically for use in field applications of ECOALERT, are provided by the invention. Typically, the deployment device is suitable for carrying one or more vessels that each comprise a semi-permeable material which allows a water sample to pass into said vessels (and is in turn typically suitable for use in the method of the invention). The kit of components is typically suitable for use in the method of the invention.

The deployment device comprises a non-watertight housing and means for securing the one or more vessels within the housing and/or means for providing flotation of the deployment device.

The kit of components comprises:

one or more vessels comprising a semi-permeable material which allows a water sample to pass therethrough;

a non-watertight carrying structure to carry the one or more vessels; and

a member for securing the position of the one or more vessels within the carrying structure and/or for providing flotation of the carrying structure.

Thus, the kit of components provided by the invention may comprise one or more vessels comprising a semi-permeable material which allows a water sample to pass therethrough and a deployment device of the invention.

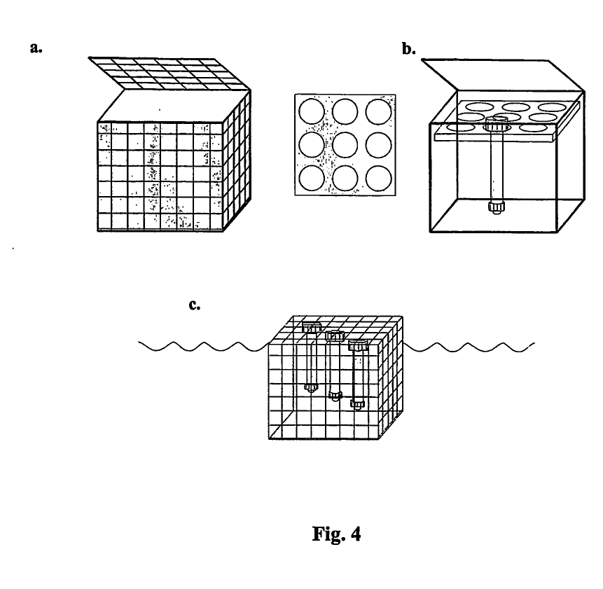

The kit of components typically comprises a suitable format of dialysis tubes to cage bacteria in open waters, a basket to deploy the dialysis tubes containing the bacteria into the water sample to be tested and a floating rack which accepts the dialysis tubes (see FIG. 4).

The one or more vessels are typically one or more dialysis tubes. The dialysis tubes may be as described above. Commercially available dialysis tubes which may be used in the kit of components include Spectra/Por® Float-A-Lyzer® and Spectra/Por® Dispodialyzer® (both from SPECTRUM).

The kit of components may comprise any number of vessels, for example 3, 6, 9, 12, 24, 48 or 96 vessels. The number of vessels required will depend on the number of replicates and blanks to be used.

The non-watertight carrying structure is any structure which is capable of accepting the one or more vessels and which allows the water sample with which the kit of components is contacted to enter so as to contact the said one or more vessels.

The non-watertight carrying structure may thus be a basket, for example a square basket. A suitable basket may, for example, be made from a plastics material or wire, such as the type used to autoclave laboratory instruments.

The non-watertight carrying structure may comprise a lid. If so, the said lid will typically be arranged so that it can be secured over the one or more vessels when the non-watertight carrying structure is loaded. Thus, the non-watertight carrying structure may comprise a fastening means to ensure that, when the lid is closed, the vessels do not float out of the non-watertight carrying structure when the kit of components is deployed in the water.

The basket must be of a sufficient height so as to accommodate the length of the one or more vessels used. The two commercially available tubes mentioned above are 10 cm and 15 cm in length respectively.

The member for securing the position of the one or more vessels within the carrying structure and/or for providing flotation of the carrying structure may be integral to or separate from the non-watertight carrying structure.

The member for securing the position of the one or more vessels within the carrying structure and/or for providing flotation of the carrying structure may be a buoyant member which provides flotation of the whole kit of components. Such a member may be ensure that the kit floats on the surface of the water sample. Alternatively, a buoyant member may be tethered to the carrying structure so that the carrying structure is suspended at a set distance below the surface of the water sample.

The member may be of suitable dimensions so that it fits inside the non-watertight carrying structure and may have one or more holes formed so as to accept the one or more vessels. The holes will typically hold dialysis tubes, if they are being used, at the neck end.

The member is therefore typically a floating tube rack formed from a buoyant material. A suitable buoyant material may be a plastics material, for example expanded polystyrene or foam.

The member may thus provide one or both of two functions: it may function to secure the position of the vessels within the basket, for example it may hold them upright; and it may function to float the entire kit of components on the surface of the water sample in which the said kit of components is to be deployed.

Two members may be provided, one of which secures the position of the vessels within the basket and one or which provides floatation. One or both of such members may be integral to or separate from the non-watertight carrying structure.

The kit of components of the invention may comprise a device for determining the optical density (and/or turbidity) of a bacterial culture and the fluorescence of a bacterial culture mixed with a redox dye. This will typically be a detection device of the sort described above.

The kit of components may comprise an incubator, typically a portable incubator, which can be set at any temperature, for example a temperature of from about 2 to about 60° C.

The kit of components may comprise means for transporting together the components of the kit. The transporting means may be, for example, a rucksack or a briefcase. Thus, the kit of components is typically portable.

The deployment device of the invention comprises a non-watertight housing and means for securing the one or more vessels within the housing and/or means for providing flotation of the deployment device. FIGS. 5 and 6 show an exemplary embodiment of the deployment device of the invention that can be used to support the vessels during sampling. Such a deployment device may be used in a kit of components of the invention.

As shown, the deployment device includes a support or rack that preferably has a plurality of locations, each for receiving one of the vessels. The locations may, as shown, simply be holes that are complementary in shape to the shape of the vessels, in which case the vessels may include a shoulder (not shown) for preventing the vessels from passing completely through the support. Alternatively the support may, for example, be formed from a resilient material such that the vessels form a tight fit when inserted in each location and are therefore held in place by friction. As a further alternative, the support may include locking means for securing the vessels in place. It will be appreciated, however, that any similar means of securing the vessels to the support may also be used.

Preferably, the deployment device includes a housing that surrounds the vessels once they are secured to the support. The housing prevents the any objects in the water being sampled from damaging the vessels. The housing includes one or more openings for allowing water to enter the housing and thence to immerse the vessels. As shown, the housing is preferably separable from the support for the vessels, allowing easy access to insert/remove the vessels from the support. It will be appreciated, however, that as an alternative, the support and the housing may be connected or form an integral unit in which case it will be necessary to provide alternative access to the support, for example a closeable hatch. As shown, the support connects to the base of the housing such that the vessels secured to the support project into the housing. In an alternative configuration, however, the support may be entirely enclosed within the housing. The support and housing may be connected to one another by means of screws, releasable clips or other fasteners, preferably made from stainless steel or other non-corrodible materials.

Conveniently and as shown, the housing may be formed from a plastics material such as PVC tubing. It will be appreciated, however, that other durable and non-corrodible materials could also be used. Similarly housings of other shapes can be used where convenient. Preferably the housing is arranged with openings on opposite sides. Consequently water can easily flow through the housing, which may help to ensure that the bacteria are in continuous contact with fresh sample. As shown, the support may be attached to one end of the housing. In this case, an opening may be provided through the support into the housing (where the vessels are located) and through the opposite end of the housing, again allowing a flow of water through the housing.

In order to ensure debris and other objects within the water to be sampled from entering the housing, any openings in the housing may be covered with a mesh. The mesh does not significantly impede the ingress of water into the housing but stops large particles from entering the housing and potentially damaging the vessels.

An attachment means may be connected to the housing. In the embodiment shown this is a narrow tube. A pole can be inserted in the tube for deploying the deployment device. Alternatively a cable or rope may be attached to housing. The housing may include weights or buoyancy sections to ensure that the deployment device has the required buoyancy and/or buoyancy distribution. For example, the deployment device may be arranged to sink in water. In this case the carrying structure may be placed on the bottom of region of water to be sampled or may be suspended at the required depth from a buoy. Alternatively, the deployment device may be arranged to float such that it can either sample the water near the surface or can be tethered or anchored at the required depth. If required, the deployment device can be configured to be less buoyant at one end than the other to ensure that it remains oriented in a given direction. For example, the support may be made of a more dense material than the housing, thereby ensuring that when the deployment device is immersed in water the support for the vessels is at the bottom.

The invention also provides a sampling device. The said sampling device is typically suitable for use in the method of the invention.

The sampling device comprises:

a sampling chamber which is adapted to accept one or more vessels comprising a semi-permeable material which allows a water sample to pass therethrough; and

a closure member selectively switchable between two or more positions, wherein:

in one of the said positions the closure member seals the sampling chamber to prevent ingress of water into the sampling chamber when the device is submerged in water; and

in a second of the said positions the closure member is open to allow ingress of water into the sampling chamber when the device is submerged in water.

The sampling device is typically adapted for sampling at a position distal from the user. In particular, the sampling device may be adapted for deployment in open water from a boat or ship. If this is the case, the sampling device may be connected to the boat or ship by a cable. The cable will of sufficient length to allow deployment of the sampling device to an appropriate depth and, if required, at an appropriate distance from the boat or ship.

The sampling device may comprise two or more, for example three, four or five, sampling chambers. If the sampling device does comprise two or more sampling chambers, the closure member may be switchable between two positions so that:

in one of the said positions the closure member seals all of the sampling chambers to prevent ingress of water into those sampling chambers when the device is submerged in water; and

in a second of the said positions the closure member is open to allow ingress of water into all of the sampling chambers when the device is submerged in water.

Alternatively, the closure member may be switchable between more than two positions such that only one of the sampling chambers is open at any time. In a device arranged in this way, the sampling chambers may be opened and closed in a sequence such that each of the sampling chambers collects a sample at a different or overlapping time points.

The sampling device of the invention may comprise two or more sampling chambers, each with a closure member, wherein the sampling chambers are arranged so that each sampling chamber is positioned at a different depth when the sampling device is submerged in water. This will allow water quality at different depths to be assessed and a profile of the water sample to be determined in terms of depth. This embodiment of then device may comprise, two three, four, five, ten or more sampling chambers, each with a closure member, wherein the sampling chambers are all positioned at different depths.

In this event, the said sampling chambers, each with a closure member, may be connected together by a cable so that the said sampling device may be deployed by lowering said cable into water to position said two or more sampling chambers at respectively different depths.

Alternatively, the sampling device of the invention may comprise two or more sampling chambers, each with a closure member, wherein the sampling chambers are arranged so that each sampling chamber is positioned at a different lateral position but at approximately the same depth when the sampling device is submerged in water. This will allow water quality at different positions in a particular water sample to be assessed. This embodiment of then device may comprise, two three, four, five, ten or more sampling chambers, each with a closure member, wherein the sampling chambers are all positioned at a different lateral position. This may be useful in the examination of, for example, an oil slick.

The sampling device may comprises one or more sampling chambers, each controlled by a first closure member and one or more further sampling chambers controlled by a second closure member. This may allow test samples to be collected from two or more depths at two or more time points with a single deployment of the sampling device.

The closure member of a sampling device of the invention may be activated by electro-mechanical means.

Where the sampling device comprises two or more sampling chambers, each with a closure member, wherein the sampling chambers are connected together by a cable, the said cable may be arranged to carry actuation signals to open or close said closure member.

In a sampling device of the invention, the closure member may comprise a plate having one or more water ingress holes therethrough and one or more index holes therethrough, said plate being moveable so as to selectively open the sampling chamber in the second position by presenting a water ingress hole over said sampling chamber. The plate may be spring-loaded and may be indexed by a solenoid actuator cooperating with said one or more index holes. A microswitch may be present to provide for feedback as to the open or closed state of the plate.

A particular embodiment of the sampling device of the invention is now described with reference to FIG. 7.

The device comprises a sampler carousel which itself comprises a sampling chamber into which one or more dialysis tubes are placed. The device is “cocked” by rotating the cap (the closure member) against the motorcycle kick-start spring until the solenoid retaining pin locates in hole 1. In this position the sampling chamber is closed. The cap is free to rotate on the shaft and is sealed at the top by “O” rings and at the bottom by a seal membrane. At deployment depth, the solenoid is fired, allowing the cap to rotate to hole position 2, where the spring pressure on the solenoid retaining pin relocates the pin. In this position, the sampling chamber is open. After expiry of the integrating period, the solenoid is again fired and the cap rotates until the pin locates in hole 3. The sampling chamber is again open in this position for sample recovery.

The sampling device has a cable of predetermined length L. A chain of sampling carousels are connected together permitting sampling of a water column depth N×L at depth interval L.

The topmost carousel is connected to a deck until for control/sensing of the sampler rig. This device will have a cable attached whose length is L+sufficient length to reach from sea surface to position on deck of vessel where deck unit is mounted.

The last carousel in the chain is connected by a short cable to a micro CTD unit (CTD=conductivity temperature depth). This device may be “up-the-wire” or self-logging. The “up-the-wire” option may be preferred because it allows positioning of the carousels in relation to water column structure, and it will be cheaper. The deck unit may be connected to a laptop for such real-time monitoring.

A self-logging CTD (they usually have a real-time up-the-wire option) will allow operation in either mode.

Each device is “cocked” by rotating cap against the motorcycle kick-start spring until solenoid retaining pin locates in hole 1. (Deck box must have push-button to fire solenoid). Chambers are closed. Micro-switch senses solenoid pin and lights “ready” indicator on deck unit. Note: Cap free to rotate on shaft, sealed at top by “O” rings, at bottom by seal membrane made of, for example, neoprene.

At deployment depth, solenoid is fired, allowing cap to rotate to hole position 2, where spring pressure on solenoid retaining pin relocates the pin. Chambers are now open. Micro-switch senses solenoid pin and lights “open” indicator on deck unit.

After expiry of integrating period, solenoid is again fired, and cap rotates until pin relocates in hole 3. Chambers are again closed for sampler recovery. Micro-switch senses solenoid pin and lights “closed” indicator on deck unit.

Correct operation of device will depend upon balance between several forces:
Rate of rotation of cap will depend on balance between strength of kick-start spring and resistance of cap to rotation.
This resistance will depend on friction of water seal against cap, and pressure of cap spring washer.
Firing cycle time of solenoid must be adjusted to be long enough for cap rotation to begin, but short enough for spring to relocate solenoid pin in next hole.
This balance will have to be determined by trial and error method in water.

The cable may have 12 cores, allocated as follows:
1 core as +12V supply;
1 core as system ground;
1 core as solenoid fire signal;
3 cores to sense each of three solenoid positions;
3 cores to address each of up to eight samplers on a string;
3 cores for RS232 Tx/Rx and signal ground for communication with CTD (if “up-the-wire” communication is used).

The following Examples illustrate the invention:

### EXAMPLES

### Example 1

### Evaluation of Growth Dynamics and Sub-Cellular Bioindicators Under (i) Non-Toxic Closed Conditions, (ii) Toxic, Closed Conditions and Assessment of Sensitivity of Bacterial Cultures to a Range of Contaminants in Closed Laboratory Experimental Systems

(i) The effect of temperature as a method to inactivate bacteria (Vibrio natriegens NCIMB 587) was investigated in order to have “dead” control samples. Two identical cultures, growing exponentially, were exposed to extreme temperatures of 5° C. and 55° C. After 24 h, both cultures were returned to the optimum growth temperature (20° C.). The 5° C. culture reactivated very fast and recovered its typical values of optical density and cell concentration. The 55° C. culture did not recover and no colonies grew when inoculated on agar plates, which indicates that bacteria were not viable after the heat treatment. A treatment of 55° C. for 24 h was thus found to be a useful method to inactivate bacteria.

(ii) Sterile, 12-ml capacity, screw-capped, clear polystyrene test tubes (Starstedt) were selected as the most appropriate closed system for use in ECOALERT experiments. These test tubes are ideal for short-term cell culturing where aeration is not critical. Also, the optical density of the culture can be read as often as desired, by simply inserting the same tube in a spectrophotometer, thus avoiding contamination risks and reducing analysis time.

(iii) The growth dynamics (growth curve, specific growth rate and doubling time) of an unidentified marine bacterium isolated from the English Channel were studied. The bacterium was isolated on LB Broth agar from an aged seawater sample and cultured in the same liquid media at 20° C. The growth rate was nearly 2-times slower than the growth rate of V. natriegens cultures grown in 210 culture media at 20° C.

(iv) The application of the 3H-leucine method of Smith and Azam (Marine Microbial. Food Webs. 6(2), 107-114, 1992) was developed and also used with 3H-thymidine to estimate bacterial production. The synthesis of DNA (thymidine uptake) and protein (leucine uptake) was studied in V. natriegens closed cultures. In non-toxic cultures the uptake of both radiotracers by the bacteria reaches maximal values between 4 and 8 hours of the experiment, coinciding with the log-phase of the growth curve.

(v) The fluorimetric measurement of the DNA specific probe DAPI (4′,6-diamidino-2-phenylindol, Sigma-Aldrich) was studied for the direct estimation of the DNA total content in aliquots of bacterial cultures was studied—no filtration is needed, which reduces handling. Until now, DAPI has been mainly applied to flow cytometry and epifluorescence microscopy. The optimal range of final concentration of DAPI in ECOALERT samples was 5-10 μg/mi. At higher concentrations either the DAPI background became too high or the sample diluted as a result of adding too much volume of DAPI solution. The optimal final concentration of DAPI had to be adjusted according to the cell density in the sample. With optical density close to or over 0.6, 10 μg/ml DAPI stained the bacteria more efficiently, while below 0.6, 5 μg/ml of DAPI was optimal. The combination of both procedures resulted in a linear relationship between DAPI signal and optical density.

(vi) The application of the redox dye CTC (5-cyano-2,3-ditolyl tetrazolium chloride, Polysciences Europe) was developed to direct fluorimetric determination of respiration in bacterial suspensions. The CTC dye produces a fluorescent, insoluble formazan when it is chemically or biologically reduced, and can be used to identify actively respiring bacteria (CTC+ bacteria). Traditionally, CTC has been used with flow cytometry and epifluorescence microscopy, combined with DAPI to counterstain total cells. However, its application to bacterioplankton remains controversial.

An exponentially growing culture of V. natrigens was incubated with the redox dye CTC following the standard procedure (2.0 mM final concentration, 4 h at 28° C.) and samples were taken every 30 minutes to count total cells and CTC+ cells in the microscope. The total cell concentration (DAPI counts) increased 3 fold during the 4 h incubation, whilst the number of CTC+ cells did not change after the first 30 min. The resulting proportion of CTC+ cells declined accordingly after the first 90 min of the incubation.

It is important to note that under the microscope the bacteria identified as CTC+ may have had formazan inclusions of varying size, from very tiny, inconspicuous spots to very large crystals which almost covered the whole bacteria. Bacteria with larger formazan inclusions radiate more fluorescence and this increases the signal measured in the fluorometer. Conversely, bacteria with small formazan inclusions will be less fluorescent and will produce a lower signal. No further study was conducted about the morphology of the intracellular formazan, although we noticed that, in general, larger formazan inclusions were formed during the stationary phase indicating increased respiration.

To avoid these shortcomings in ECOALERT experiments, the following criteria was adopted: i) CTC incubations were adjusted to 2 hours ii) the CTC+ fluorescence signal was compared only within samples of a single experiment or used as a qualitative estimator of respiration between experiments iii) the DAPI counts from the CTC microscopy samples were only used to calculate the % of CTC+ bacteria, but not as an estimator of cell concentration during the experiment.

(vii) The growth dynamics and sub-cellular bioindicators of a closed culture of V. natriegens during a long-term experiment (38 days) were studied to see the effect of ageing and nutrient depletion.

For this experiment, a large polypropylene container was filled with nearly 2 litres of bacterial culture and incubated with agitation. No aeration was provided though, so bacteria might have experienced oxygen limitation, besides nutrient limitation, and their metabolism might have been affected.

After 38 days, cell concentration reached 2.7×10⁹ cells/ml. Growth rate declined exponentially. Respiration per cell, estimated as the ratio CTC fluorescence/optical density also declined steadily early in the experiment. Thymidine and leucine incorporation followed very closed one to other, but thymidine incorporation increased after 100 h suggesting that DNA synthesis was enhanced relative to protein synthesis.

(viii) Two ready-to-use commercial kits to assess cell viability (ProCheck™ Cell Viability Assay) and membrane integrity (LIVE/DEAD® BacLight™ Bacterial Viability Kit) were tested and adapted for using during ECOALERT experiments.

ProCheck™ is designed to measure the amount of viable, metabolising cells in a sample, and LIVE/DEAD® BacLight™ distinguish live and dead bacteria As a preliminary test, the two kits were tested in a volumetric series of mixtures of two bacterial types of V. natriegens: live, exponentially growing bacteria and dead bacteria killed by autoclaving an aliquot of the same culture. The colorimetric signal indicating cell viability (ProCheck™) and the fluorimetric signal indicating membrane integrity (LIVE/DEAD® BacLight™) increased linearly with the proportion of live bacteria in the mixture.

In a second experiment, the viability kits were assessed with four contaminants: pyrene (pyrogenic hydrocarbon), copper (metal, algaecide), dodecylbenzensulfonic acid sodium salt (LAS type of anionic surfactant, industrial detergent) and TBBA (flame retardant), against a non-toxic control.

All toxic cultures experienced membrane damage after 8 h compared with the non-toxic control, as detected by LIVE/DEAD® BacLight™ assay. In contrast, with the exception of Cu, ProCheck™ did not revealed any clear effect in any other toxic culture.

Compared to the non-toxic control, and even to other contaminants, Cu had the most deleterious effect causing a notable slow down of the growth curve and affecting cell viability.

The effect of the detergent on the bacteria was complex. Firstly, the growth of the culture increased over the control after 8 h, and secondly the LIVE/DEAD® BacLight™ test indicated a progressive damage of cellular membranes with time. Nevertheless, it should be noticed that the LAS surfactant used in our experiments gave high absorbance and fluorescence blanks. The addition of detergent to culture media-only (final concentration 0.1 g/l) increased the absorbance 95 times, and also increased the fluorescence 1.5 times. These optical properties may have biased the results.

(viii) The effect of a range of contaminants on growth dynamics and sub-cellular bioindicators in closed cultures of V. natriegens was studied:

Exposure to phenanthrene (hydrocarbon) showed no concentration-related effect, so that the results from the three concentration treatments were averaged for simplification. Phenanthrene did not affect bacterial growth significantly. Compared to the control, contaminated bacteria doubled their initial rate of protein synthesis and DNA synthesis rates where on average 30% higher. No effect was observed on respiration.

Pyrene (pyrogenic hydrocarbon) caused an average 14% increase in respiration, but no other effect was observed.

Lindane (pesticide) appeared to cause a transient, rather subtle stimulation of bacterial growth rate and respiration that, however, normalised after approximately 10 hours.

The symptoms of stress by copper were extreme: all cellular activity (growth, respiration and DNA/protein synthesis) nearly ceased immediately after the inoculation.

Complex changes occurred during the exposure to the detergent. After an instant 10-fold reduction in bacterial numbers and respiration, both indicators increased, though bacterial numbers did not reach those of control. Basically, the overall tendency of respiration was contrary to that of the non-toxic control. The synthesis of protein by bacteria was inhibited by the detergent throughout the experiment but seemed to recover at the end of the 72 h reaching control values, whilst DNA synthesis increased slightly over the control in the first 24 hours. The relationship between optical density and cell concentration suggests that bacteria progressively reduced their size in the presence of the surfactant.

The addition of TBBA (flame retardant) caused an initial increase in growth rate, though the optical density and the cell concentration did not show major difference compared to the control. However, TBBA clearly affected respiration of bacteria because after the 4th hour there was a sudden and important increase in respiration, and the ratio respiration/optical density indicated an average increase of more than 90% in cellular respiration. This is likely to indicate that the cells were severely stressed. There are no data about DNA/protein synthesis in the last 6 hours (samples were contaminated), but it is likely that these rates also increased over the non-toxic control.

(ix) The growth of caged V. natriegens in dialysis bags was compared to the evolution of the same culture in a closed system.

The growth dynamics of bacteria in dialysis bags (Slide-A-Lyzer® 10K Dialysis Cassettes) was compared to closed test tubes. Two dialysis cassettes and two test tubes, were filled with 10 ml of a fresh culture of V. natriegens and incubated in a 20 L tank of pre-filtered (1 μm) seawater at 18° C. At 1-h intervals the optical densities of the bacterial suspensions were read and aliquots taken for cell counting. After 12 h, the bacteria caged in the dialysis bags had reached only half of the optical density and cell numbers of those closed in the test tubes, most likely due to the loss of the nutrients by diffusion through the dialysis membrane. The direct counts also showed that the caged bacteria soon became smaller than the closed ones. This is probably why the caged samples yielded higher bacteria concentration than the closed ones for any given absorbance value, though the dilution of the culture media through the dialysis membrane may also have affected the optical density of the culture.

The average specific growth rate in the dialysis bags was 0.49 l/h, which is not far from the 0.57 l/h calculated in the closed test tubes. The fact that cell growth still occurred rapidly with the dialysis bags was very promising and indicated that the ECOALERT concept of using “caged” bacteria was viable.

(x) The sensitivity of the conformation of the cell wall of bacteria, investigated through lectin binding was studied as a sub-cellular bioindicators of toxicity.

Bacteria were reacted with two fluorescent lectins prior to- and post-toxic exposure to test the hypothesis that toxicity induces changes in the conformation of the cell wall, resulting in an alteration in lectin induced fluorescence. The results were inconclusive and there was no clear difference in the fluorescent signal attributed to toxic exposure due to considerable non-specific binding and background staining.

(xi) The sensitivity of cultures of V. natriegens to a range of contaminants in closed laboratory experimental systems was studied at specified toxic concentrations and time scales.

Sensitivity to Phenanthrene (Hydrocarbon).

There is no major effect on bacterial growth or metabolism, apart from a small initial stimulation of the synthesis rates of protein.

Sensitivity to Pyrene (Pyrogenic Hydrocarbon).

The only detectable effect is a relative increase of bacterial respiration and loss of membrane integrity in the stationary phase.

Sensitivity to Lindane (Chlorinated Pesticide).

Lindane causes a transient, faint stimulation of bacterial growth rate and respiration during the log-phase.

Sensitivity to TBBA (Flame Retardant).

The most evident toxic effect of TBBA is a notable increase of respiration after 4 hours of exposure, which indicates stress. Also, TBBA causes membrane damage in the stationary phase.

Sensitivity to Copper (Metal, Algicide)

Cu seriously affects bacterial growth and metabolic functions causing a very conspicuous and instantaneous reduction in all the studied bioindicators compared to non-toxic conditions. Besides, Cu causes a damage of membranes similar to the other contaminants tested.

Sensitivity to LAS Anionic Surfactant (Industrial Detergent)

After an initial adverse effect, the detergent seems to enhance growth and respiration. Protein synthesis is inhibited but seems to recover after 48 h. The size of bacteria reduces compared to the control bacteria. Finally, there is a deleterious effect on membranes during the stationary phase. It is likely that detergent is bio-degraded by the bacteria. There is evidence of bacterial degradation of LAS surfactants in estuaries, the efficiency of this biodegradation strongly depending upon salinity. The utilisation of the detergent by bacteria would explain the observed acceleration of the growth curve, the increase of respiration and the final recovery of the protein synthesis rate.

The simultaneous investigation of several bioindicators is an advantage for ECOALERT because it gives a more complete diagnosis of the status of a bacterial culture and, in consequence, makes it possible to discriminate the toxic effect of a range of contaminants. The bioindicators that we selected proved very useful: their experimental methods were optimised for time, instrumentation and costs, and when used to assess bacterial cultures they drew different diagnostics for every contaminant.

### Example 2

### Evaluation of Different Dialysis Systems, Study of Growth Dynamics Under Caged Conditions and Evaluation of Growth Dynamics and Sub-Cellular Bioindicators Under (i) Caged, Non-Toxic Laboratory Conditions and (ii) Toxic, Caged Laboratory Systems

(i) Two commercial dialysis systems were evaluated: a cassette slide system (Slide-A-Lyzer®, Pierce) and a tube system (Spectra/Por® Float-A-Lyzer®, Spectrum).

Both dialysis systems were evaluated during several trials in 2-litre and in mesocosm tanks. The ready-to-use dialysis tubes from Spectrum were the most satisfactory and seemed the best design to be used in future ECOALERT field tests.

(ii) The marine bacterium Pseudomonas sp. (strain 1534) obtained from the National Collection of Industrial and Marine Bacteria (NCIMB, Aberdeen) was chosen as the new test organism for ECOALERT experiments. This is a gram negative bacterium and was originally isolated from seawater in the North Sea. A permanent stock is kept at Plymouth Marine Laboratory in 15% glycerol-seawater yeast peptone medium (210 NCIMB medium) at −80° C. Temporary stocks for ECOALERT experiments may be kept at 4° C. streaked in 1.2% agar-210 NCIMB medium and renewed periodically. Some of the ECOALERT experiments were carried out using Vibrio natriegens (strain 857). This is a gram negative bacterium also obtained from NCIMB and kept as temporary stocks at PML in the same fashion as described above for Pseudomonas. The original strain was isolated from salt marsh muds in Georgia, USA.

Pseudomonas sp. is idoneous for ECOALERT purposes for a number of reasons:

The Pseudomonas strain used was isolated from the North Sea.

Pseudomonas sp. can grow as rapidly as Vibrio natriegens, which was used during former ECOALERT experiments, and seems to adapt even better to caged systems.

Pseudomonas sp. tolerates a range of salinity representative of the gradient from open shelf to estuarine waters. The growth rate is constant within the gradient 20-35, and below salinity 10 the growth rate decreases dramatically.

The sensitivity of Pseudomonas sp. to contaminants is very similar to that observed for Vibrio natriegens.

(iii) In order to improve the sensitivity, rapidity and ecological relevance of ECOALERT toxicity assays, some changes were introduced in the experimental design:

The standard experimental temperature was set to 15° C. instead of 20° C., to approach natural conditions.

For the bacterial assays, a diluted culture medium was used. This medium was prepared sterile with 210 NCIMB (seawater yeast peptone) medium 10-times diluted in 3/4 seawater (0.2-μm filtered) and 1/4 distilled water. This diluted medium reduced the growth rate of the bacteria, but also minimised the chemical complexation of some contaminants thus increasing the sensitivity of the bacterial assay to toxicity. On the other hand, diluting the culture medium improved all optical and fluorescent readings by lowering the background values of the medium blanks.

The optical density of the bacterial cultures was measured at 540 nm wavelength (OD₅₀) instead of 595 nm.

(iv) Experimental set-up for caged laboratory systems.

On the night before the experiment, a number of 2-litre HDPE bottles, one bottle per toxicity treatment, were filled with water (3/4 seawater 1-μm filtered, 1/4 distilled water). The bottles were kept at 15° C. stirring at low speed.

The dialysis tubes, which are supplied soaked in sodium azide, were rinsed throughly, refilled with distilled water and UV-irradiated for 10 minutes to ensure sterility.

Also the night before, a fresh culture of bacteria was inoculated from the original stock in liquid diluted medium.

Before starting the toxicity assay, the bottles were spiked with the contaminant and buffered to pH 7.3. At the same time, a fresh volume of diluted medium was inoculated from the culture grown overnight and kept for ˜2 hours at 15° C. until the growth rate picked up again and OD₅₄₀ approached 0.005.

Then, the dialysis tubes were emptied of the water and four of them topped up with the bacterial culture, plus one blank tube with medium-only (no bacteria), and placed into the bottle to start the assay. Each bottle could hold 5 dialysis tubes floating upright.

The stirring was kept low and at time intervals samples were removed from the tubes with sterile pipettes to measure the different bacterial indicators.

(v) To estimate bacterial numbers from OD values, a conversion factor was determined. A sample of a Pseudomonas sp. culture was sequentially diluted 10 times, measuring the OD values and counting the bacterial numbers for each dilution. According to the regression bacterial numbers v OD, abundance could be estimated as a polynomial function of OD₅₄₀: abundance (10⁹ cells/ml)=1.8845OD²+0.9552OD.

(vi) Four bacterial sub-cellular indicators were investigated: DNA, respiration, membrane integrity and viability. These indicators were chosen because they represent bacterial parameters closely related to metabolism and growth, and could be determined directly in cellular suspensions with fluorescence and colorimetry techniques using commercial probes and reagents.

Bacterial DNA. To improve the detection of bacterial DNA in culture samples, the PicoGreen double-strand DNA (dsDNA) reagent (Molecular Probes) was used instead of DAPI. PicoGreen reagent is more sensitive to small changes in the DNA content of bacterial cultures, resulting in a good correlation with OD.

Respiration. Respiration was estimated with the redox dye CTC (Polysciences) as values of fluorescent intracellular formazan. Using diluted culture medium greatly improved the measure of this indicator.

Integrity of bacterial membranes. The membrane integrity was evaluated with the LIVE/DEAD® BacLight™ fluorescence kit (Molecular Probes), with a slight adaptation of the manufacturer instructions.

Cell viability. The viability of the bacteria was assessed applying the colorimetric ProCheck™ assay (Intergen).

(vii) The growth dynamics of non-toxic cultures of caged Pseudomonas sp were studied. The growth of Pseudomonas sp. in dialysis cages during the first 8-10 hours generally described an exponential curve. The final OD reached depended largely on the initial cell density set at the start of the incubation. During a 10-hours cage assay in non-toxic conditions OD generally increased 10 times.

In ECOALERT cage assays, the initial OD in the dialysis chamber was set approximately to 0.005. Converted to bacterial abundance, this was about 5×10⁶ cells/ml and within the range found in seawater. Ideally, after 10 hours at 15° C., the bacterial abundance in the chamber reached approximately 50×10⁶ cells/ml (=0.050 OD).

(viii) The sub-cellular indicators in non-toxic cultures of caged Pseudomonas sp. were studied in relation to the growth curve.

The content of DNA was closely related to the optical density of the sample and the regression described a highly significant exponential curve. If bacterial division is in phase during these assays, then the fluorescence signal detected picked up the synthesis of new DNA and the changes in the condensation of the nucleoid that precedes bacterial division. These were reflected in that exponential relationship with OD.

Bacterial respiration increased with time and was also significantly and positively related to OD. The redox dye CTC was taken up and reduced to fluorescent formazan by active cells, so this respiration indicator must have increased proportionally with bacterial abundance. There was a certain degree of variation of the respiration value for a given OD, which could be related to changes in the physiological state of the cells during exponential growth.

The results from the indicator of membrane integrity were unclear and highly variable. No relationship was found with any other cellular indicator.

The bacterial viability showed no consistent results either and no relation with growth.
(ix) The growth dynamics of toxic cultures of caged Pseudomonas sp. were studied. The effect of toxicity on the growth of caged bacteria was studied in several assays where different concentrations of contaminants were added to the water of the tanks. The sensitivity to toxicity in each assay was measured as the limitation of growth, estimated as OD, relative to the metal-free control calculated following Kahru et al. (Water Science and Technology 33, 6, 147-154, 1996):

$$KF = \frac{{IC}_{t}}{{IC}_{0}} ; Limitation \% = 100 - \frac{{IT}_{t}}{KF \times {IT}_{0}} \times 100$$

where IC₀ and IT₀ are the initial values of OD of metal-free control and test samples, and ICₜ and ITₜ are the values measured at t time.

The hazardous substances tested: heavy metals and a household disinfectant, are common in polluted coastal waters: cadmium, copper, chromium, mercury, nickel, lead, zinc and chloroxylenol (Dettol).

Effect of cadmium. The toxic effect of Cd was detected as a 59% growth limitation at 20 mg Cd/L treatment after 4 hours of exposure. The lower Cd treatments did not cause any clear limitation of bacterial growth.

Effect of copper. Three assays were run testing different ranges of Cu concentrations. During assays 1 (doses 0.001-10 mg Cu/L) and 2 (doses 0.02-20 mg Cu/L) only a low limitation (<50%) of growth was observed. Additions under 0.1 mg/L had an enhancing effect on growth, which may be due to requirement of uptake of trace nutrients by the bacteria. During the third assay, however, 56% growth limitation was measured at 20 mg/L after 9 hours.

Effect of mercury. The toxicity of Hg was rapidly detected in the caged bacteria during two different assays. Up to 60% growth limitation occurred in the first 4 hours of exposure at concentrations as low as 2 mg/L.

Effect of nickel. At the end of a 9-h assay, 51% growth limitation was measured at 5 mg/L and 69% at 20 mg/L. Below 0.5 mg/L, Ni seemed to favour growth.

Effect of zinc. No significant toxicity was observed in a first experiment with doses between 0.05-5 mg/L. On the contrary, zinc had a positive effect in most of the treatments. During the second assay using higher concentrations, however, growth limitation reached 51% after 4 h at 50 mg/L.

Effect of lead. During two lead assays, growth limitation reached 52% at 2 mg Pb/L after 4 h, and this toxic effect steadily increased with time and metal concentration.

Effect of a metal sludge cocktail containing proportions of the heavy metals Cd, Cu, Cr, Ni, Pb, Hg and Zn. In this assay, three different “pollution level” treatments were prepared based on the metal proportions specified in a municipal metal sludge standard recipe from BDH. The treatment level “2” caused 51% growth reduction of the caged bacteria after 6 h. At the pollution level “3”, the growth limitation reached 79%. The results from this experiment must be interpreted cautiously because the initial t=0 samples were taken well in advance of the experiment and that could have affected the apparent sensitivity.

Effect of the antiseptic disinfectant Dettol (main active ingredient, phenolic biocidal chloroxylenol 4.8% w/v). The three concentrations tested were highly toxic and caused growth limitation over 90% after 5 h of exposure. These doses of Dettol were based on the manufacturer recommended dilution for most household and medical uses, so this experiment must be taken as a preliminary test on potential source concentrations.

(x) Bacterial sub-cellular indicators (DNA, respiration, membrane integrity and viability) were studied in toxic cultures of caged Pseudomonas sp.

Changes in the quantity of bacterial DNA were studied during different toxicity assays. The DNA indicator is highly correlated with OD. The x coefficient of the regression DNA fluorescence v OD seems to be very conservative within the single metals assays (mean=5.17±0.36 S.D.), but the meaning of this coincidence was not totally clear; possibly it indicated a similar mechanism of toxicity. DNA was a sensitive indicator of toxicity for Dettol, Zn and metal sludge, but could not detect limitation during the Cu assay.

Respiration was also well correlated with OD. The x coefficients of the regressions v OD during single metal assays were, again, very similar (mean=0.77±0.10 S.D.). Respiration was a sensitive and early indicator of toxicity in most cases. In particular, respiration was the most sensitive indicator of toxicity for exposure to Cd, Hg and Pb.

The viability and membrane integrity indicators were inconclusive. The only clear results were seen during the metal sludge assay. The viability indicator appeared to be the most sensitive during this assay detecting a 72% reduction in bacterial viability at the pollution level “1”. During all the other toxicity assays the results were highly variable and showed no relationship with growth.

### Example 3

### Evaluation of Key Sub-Cellular Bio-Indicators in Laboratory Experimental Systems, Selection of Appropriate Bio-Indicators, Production of Detailed, Standardised Procedures for Selected Bio-Indicator Methods, Development and Testing of Field Application System in Mesocosm and Development of Clean Water Test System as Control

1. Evaluation of Bacterial Endpoints in Laboratory Systems

Two bacterial endpoints were chosen based on the results from Example 2: growth and respiration. Growth is an endpoint at population level and is estimated by measuring in vivo optical density (OD) or turbidity (TRB). Respiration is an endpoint at subcellular level and is measured by the fluorescence emitted by actively respiring bacteria, using the CTC redox dye.

To assess OD, TRB and CTC as indicators of toxicity a series of ECOALERT assays were carried out on a wide range of chemical substances, with special attention to common pharmaceuticals but also including some contaminants cited in the OSPAR list for priority action (OSPAR 2000). The concentrations tested were based on toxicological and environmental levels, where this information was available.

The set-up conditions for these assays were modified as necessary, with the aim of testing the ECOALERT method and the sensitivity of the indicators. A summary of the experimental conditions that were tried is given in Table 1.

TABLE 1
Range of set-up conditions tried in order to test
the behaviour of the bacterial indicators and to
optimise the ECOALERT toxic assay method.

| | |
|---|---|
| Experimental | closed system: test tube |
| systems | open system: dialysis tube |
| | floating in tanks |
| Tank sizes | 2 litres |
| | 20 litres |
| | 200 litres |
| Water system | contained tanks |
| | flow-through system |
| Experimental | between 7 and 22° C. |
| temperature | |
| Initial density | between 0.002 OD and 1.0 OD |
| of bacteria | |
| Exposure | between 6 and 72 hours |
| Detection of growth | bench-top spectrophotometer |
| | to measure OD |
| | portable turbidity meter to |
| | measure TRB |
| Temperature during | same as experimental temperature |
| incubation of | 28° C. |
| bacterial samples | |
| with the CTC dye | |
| Duration of | 0.5 hour |
| incubation of | 1 hour |
| bacterial samples | 2 hours |
| with the | |
| CTC dye | |
| Detection of | bench-top fluorometer to measure |
| respiration | CTC fluorescence |
| | portable standard fluorometer with |
| | fixed chlorophyll channel |
| | portable adapted fluorometer with |
| | CTC channel |

A cell viability indicator using the ProCheck reagent (see Example 2) was tested in one experiment but not considered further because the results were not consistent and the method imposed some complications for a field application. For example, the reagent needs to be stored at −20° C. and thawed just before use.

The other two sub-cellular indicators examined during the assays were membrane integrity (LIVE/DEAD® BacLight™) and bacterial DNA (PicoGreen), but both of them have been considered impractical for use in the field: LIVE/DEAD® is a mixture reagent that needs to be prepared from two components, centrifuged and added to the bacterial suspension in a cell-density dependant proportion for the method to be accurate, while the PicoGreen reagent needs to be diluted in a DNA-free buffer solution and is very susceptible to photodegradation.

1.1. Bacterial Endpoints Finally Selected for ECOALERT

1.1.1. Growth Indicators OD and TRB

Optical absorbance was measured as the absorbance of a live sample of bacterial culture in a spectrophotometer set at 540 nm wavelength. Depending on the density of the bacterial culture at the start of the assay, and also on the time length of the exposure to the chemical substance, OD did not always show a clear effect in the exposure treatments during the first 24 hours. In other cases, the effect of a certain dose of contaminant on OD changed dramatically from positive to negative after 24 hours. The best results for this indicator were achieved when the assay was started with a growing culture of bacteria at approximately 0.04 OD.

To test the effect of the initial OD on the growth rate, a simple experiment was carried out setting four cultures at four different initial OD. The four cultures were grown for 6 hours in dialysis tubes floating in natural seawater, and samples were taken at intervals to measure OD and calculate the growth rate.

The results showed that the highest exponential growth rate was achieved in the culture started at 0.04 OD.

TRB is another indicator of bacterial growth in vivo, just like OD, although it was measured with a turbidimeter. The advantage of using the indicator TRB is that, in the field, it can be measured simultaneously in the same sample with the same instrument as CTC, using a Turner Designs prototype of portable fluorometer/turbidimeter. The behaviour of TRB was very similar to OD and both indicators were closely correlated and could be accurately inter-converted for the same sample.

1.1.2. Respiration Indicator CTC

Respiration activity in the bacterial culture was estimated using the CTC redox dye by measuring the fluorescence emitted by the fluorescent formazan product of CTC, reduced by the respiring cells. The fluorescence could be measured in a fluorometer set at 488 nm emission and 630 nm excitation wavelengths. In most of the experiments, the sub-cellular indicator of bacterial respiration CTC showed significant responses within the first 24 hours of exposure to the chemical.

CTC was an early indicator of effect as compared to the growth indicators OD and TRB, particularly in those cases where the effect was detected in the later hours of an experiment. One possible explanation was that during the stationary phase, once bacterial growth slowed down, bacterial number may not have been affected so much by the presence of a chemical, even if this was causing mortality, and then no change would have been seen in either OD or TRB. However, the effect of the chemical may have been detected by the respiration indicator CTC.

1.1.3. Ratio Respiration/Growth

Even when no clear effect could be observed in the CTC indicator alone, there was an advantage in using the bacterial indicators OD (or TRB) and CTC simultaneously. That was because it was possible to use the ratio respiration/growth, which is an estimator of the proportion of respiring or active bacteria in the population, as an indirect indicator of the health of the population. In some experiments a given dose of the chemical did not cause any significant effect on the bacteria compared to the non-toxic control treatment and no changes in growth or respiration could be measured.

The study of the CTC/OD and CTC/TRB ratio did, however, give some additional information. For example, in the case of the carbamazepine test there was an initial positive effect on the CTC/TRB ratio that comes down to control values after 24 hours, although the trend suggested that there could be a negative effect in a longer exposure. In the case of PCP, at the end of the exposure to PCP the CTC/TRB ratio confirmed that PCP caused a loss of respiring cells in the culture compared to the control.

In non-toxic conditions, the number of respiring cells in a bacterial culture was a fixed proportion of the total number of cells within the first 24 hours of the caged bacterial culture being transplanted into natural water. This was confirmed by plotting CTC against OD, using data pooled from the non-toxic treatments of different experiments. CTC and OD were measured on the same sample after incubating the bacteria with the CTC reagent during 1 hour.

These results support the use of OD (or TRB) and CTC during ECOALERT assays because combined, the two bacterial indicators growth and respiration can be indicatives of the mechanisms of action of the chemical substance on the bacteria. The indicators OD, TRB and CTC can also detect positive effects in situations when the chemical stimulates growth and/or respiration of bacteria. This has important implications for the natural environment, for example regarding the balance between CO₂ production and O₂ consumption in the water.

### Example 4

### Standardised Procedure for the Determination of OD, TRB and CTC During ECOALERT Assays

A Standard Operational Procedure (SOP) has been developed as a guidance for ECOALERT assays in meso-scale laboratory systems. This SOP is based on the two bacterial endpoints finally selected: growth and respiration.

1.1. Assay System

The assay of the invention typically uses a strain of marine bacteria isolated from North Sea waters and is designed to test toxicity in seawater in mesoscale systems simulating natural conditions. The assay tests two variables in the bacteria: growth and respiration. The growth rate of the bacteria is estimated measuring the optical density of the culture. The proportion of respiring bacteria at any given time may be estimated by staining the culture sample with a fluorescent redox dye and measuring the fluorescence on a fluorometer.

1.2. Response Criteria

Two parameters are used to estimate the response of the bacterial culture to toxicity: growth and proportion of respiring (active) cells.

1.3. Duration of Assay

The actual assay is optimised for 24 hours but it can be extended or shortened if required. For a 24 hours assay, samples are taken at 0, 6 and 24 hours and the whole procedure takes 3 days. Alternatively, samples can be taken hourly during about the first 8 to 10 hours, plus additional samples at 24, 28 or 72 hours.

1.4. Basis of Method

The method uses marine bacteria to determine the toxicity of seawater and it is based on the fact that bacterial activity and growth will be affected negatively by toxic substances present in the test water. The extent of the negative effect is measurable and can be related to the concentration of toxic substances.

1.5. Range of Application

The method can be applied to test toxicity in mesoscale systems that simulate conditions ranging from the open sea to low salinity, estuarine waters.

2. Principle

The method is based on the concept of “caged” bacteria, which can be transplanted into the test water and the response of the bacterial culture followed. A particular format of dialysis tubes are used as “cages”. The dialysis membrane allows the flow-through of water with dissolved nutrients and toxic substances while keeps the bacteria inside. After a period of time, the bacteria can be harvested and analysed to determine the potential effect of the toxic substances present in the water.

3. Scope and Limitations

The method can be used to test any type of seawater. The only limitations of this assay are given by the capability of this particular strain of bacteria to grow in extreme conditions of temperature and salinity, e.g. the grow rate of these bacteria slows down below 10% salinity and about 7° C.

4. Information on the Test Substance

Prior to carrying out the assay, it is recommended that information is complied about the test substance or substances such as environmental levels, toxicological data, solubility, stability, etc., and that the assay is designed accordingly (e.g. exposure concentrations).

5. Hazards/Precautions

5.1. It is mandatory to follow instructions from manufacturer, material data sheet, COSHH requirements, etc. for storage, handling and use of any hazardous chemical.

5.2. Use gloves and lab coat.

5.3. If insoluble substances are to be tested, they may be made into solutions using organic solvents such as ethanol, methanol, acetone, DMSO etc. (see manufacturer's instructions and material data sheet information). In these cases, it is recommended to set additional control treatment tanks where the solvent-only is added in the same concentration in order to determine its possible effect on the bacteria The solvents used must be of high purity grade.

5.4. On completion of the assay, all residual cultures and used consumables should be autoclaved and containers etc. well rinsed before disposing. For disposal of the test water, it is recommended to consult with staff in charge of the facilities.

5.5. The bacterial cultures should be manipulated using sterile flasks, pipettes, cuvettes, etc. discarded after single use. Inoculate the initial cultures in a laminar flow cabinet or close to a flame (Bunsen burner) to prevent contamination.

5.6. The bacteria have been tested for the ranges 7-22° C. temperature and 10.0-35.5% salinity. If the experimental water is out of these ranges, the growth rate of bacteria should be tested in non-toxic conditions prior to the toxicity assay.

6. Source and Culturing of Bacteria

6.1 Most of the assays were done using Pseudomonas sp. (strain 1534) obtained from the National Collection of Industrial and Marine Bacterial (NCIMB, Aberdeen). This is a gram negative bacterium and was originally isolated from seawater in the North Sea. A permanent stock is kept at Plymouth Marine Laboratory in 15% glycerol-seawater yeast peptone medium (210 NCIMB medium) at −80° C. Temporary stocks for the experiments are kept at 4° C. streaked in 1.2% agar-210 NCIMB medium, and renewed periodically. Some of the assays were done using Vibrio natriegens (strain 857) also obtained from NCIMB and kept as temporary stocks at PML in the same fashion.

6.2. To make 1 litre of 210 NCIMB liquid culture medium add 3 g peptone+5 g yeast extract+750 ml seawater+250 ml distilled water, adjust pH to 7.3 (NaOH, HCl), filter through GF/F and autoclave.

6.3. To make 210 NCIMB agar culture medium, follow the recipe above and add 12 g of agar per litre (1.2% agar) before autoclaving.

6.4. For the assay of the invention, use a 1:10 dilution of the 210 NCIMB liquid culture medium, prepared in the following manner: 100 ml medium+675 ml seawater+225 ml distilled water.

6.5. The seawater used to make up culture medium has been “aged”, for example left in a closed container in the dark for at least one week before used, and filtered through 0.2 μm.

7. Apparatus

Temperature controlled incubator (Thermotote de luxe portable incubator, M-TECH Diagnosis Ltd.)

Spectrophotomer (set at 540 nm wavelength)

Fluorometer (set at 488 nm excitation, 630 nm emission wavelength)

Magnetic stirrers and PFE stirring bars, autoclaved

Tanks, moulded blown glass, 20 L

Laminar flow cabinet with UV lamp

Bunsen gas burner

pH meter

Hand refractometer to measure salinity

Filtering system

Autoclave

Micro-balance

8. Consumables

Ready-to-use dialysis tubes Spectra/Por® (SPECTRUM with regenerated cellulose membrane, in two formats: Float-A-Lyzer®, 10 ml volume, snap cap, 60 kDa MWCO; and DispoDialyzer®, 5 ml volume, screw cap, 25 kDa MWCO.

CTC redox dye (5-cyano-2,3-ditolyl tetrazolium chloride, Polysciences)

Bacto™ Peptone (DIFCO)

Bacto agar™ (DIFCO)

Yeast extract (DIFCO)

19M NaOH

37% HCl

Culture flasks, sterile, conical, 500 ml

Culture flask, sterile, conical, 100 ml

Autoclavable bottle to prepare culture medium, 2 litre

Measuring cylinder

Sterile venting closure BugStopper™, autoclavable (WHATMAN)

Microbiological wire loop

Fluorometer cuvettes, polystyrene, disposable, 3 ml (SIGMA)

Extra long Pasteur pipette, disposable, 6 ml (VWR International), UV radiated

Weighting boats for the micro-balance

GF/F filters

Membrane filters, 0.2 μm

Powder free gloves

Tissues (Kimwipes®, Kimberley Clark)

9. Experimental Procedure

9.1. Day 1

9.1.1. Fill the experimental tanks (1 clean control tank+n exposure tanks) with non-toxic seawater. Tank volumes may range from 20 to 200 litres for meso-scale laboratory experiments. The tanks should be provided with agitation or aeration and will be left overnight at the experimental temperature. Alternatively, in the case of a flow-through system, connect the tanks to the reservoirs of contaminated water and/or test substances so that the chemostat can be stabilised before the start of the assay. Note that in flow-through tanks the free-floating dialysis bags can block the water outlets and provision should be made to reduce this risk by placing a mesh over the outlets.

9.1.2. Fill a sterile, 100 ml culture flask with 50 ml of the 1:10 diluted medium and inoculate with bacteria from the agar stock with a flamed microbiological wire loop. Leave this preculture overnight at the experimental temperature, closed with a venting sterile stopper and gently stirred (200 rpm).

9.1.3. As the dialysis tubes are shipped in a preservative solution they should be thoroughly rinsed prior to use with clean distilled water. Fill up each tube and its protective plastic case with clean distilled water, place them under the UV light for a few minutes and store them cold (4° C.) until next day. For any assay, it is recommended to triplicate the dialysis tubes per treatment and time point. Additionally, prepare two “blank” tubes per tank, which will be filled with media-only, without bacteria. An assay consisting on 4 treatments, 3 time points, 3 replicates and 2 “blanks” per tank will require (4×3×3)+(4×2)=44 dialysis tubes.

9.1.4. Each dialysis tube holds 10+ ml or 5+ ml depending on the format of dialysis tube (Float-A-Lyzer® or DispoDialyzer® respectively). Based on the number of dialysis tubes required for the assay, prepare two sterilised culture flasks (500 ml) with enough volume of 1:10 diluted medium to fill the sample dialysis tubes and the “blank” dialysis tubes, respectively. The two culture flasks are kept at the experimental temperature overnight, closed with ventilating stoppers.

9.2. Day 2

9.2.1. Add the test substances into the tanks. In the case of a flow-through system the contaminants should be already circulating in the tanks.

9.2.2. Use the 50 ml of bacterial culture grown overnight to inoculate the 500 ml sample culture flask so that optical density measured in the spectrophotometer (wavelength 540 nm) approaches 0.02. The 500 ml of culture will be used to fill the sample dialysis tubes for the assay, once it enters the exponential growth phase. To that end, the optical density (OD) will be checked regularly during the next 1 or 2 hours. Optimally, the assay should start when the bacteria reach 0.040 OD.

9.2.3. Prepare CTC solution. Dissolve 100 mg CTC in 6.6 ml of ultra-pure Milli-Q water. The solution is filtered through a 0.2-μm syringe filter, stored in the dark and kept refrigerated between uses. A volume of 6.6 ml CTC solution will stain 60+ samples (final concentration in sample 2.5 mM CTC).

9.2.4. Fill dialysis tubes with bacteria and start assay. Empty the distilled water from the dialysis tubes before filling them with the bacterial culture (OD˜0.040). The “blank” dialysis tubes are also filled with from the medium-only culture flask. “Blank” dialysis tubes should be labelled to distinguish them from the dialysis tubes containing bacteria

9.2.5. The assay starts when all the dialysis tubes are floating in the tanks. Annotate the start time, along with water temperature, pH and salinity in each tank.

9.2.6. Sampling. Time 0 should be sampled immediately after the assay starts. To do so, gently and thoroughly mix the content of each dialysis tube with a Pasteur pipette and remove 1.9-ml into a fluorometer cuvette to read the OD in the spectrophotometer. Immediately, add 100 μl of CTC solution into each cuvette and incubate during 1 hour at 28° C., protected from the light. Later, the fluorescence should be measured in the fluorometer (excitation 488 nm, emission 630 μm) and recorded. OD may be read again and recorded. The two “blank” tubes from each tank should be sampled and processed in the same manner as the tubes containing bacteria.

9.2.7. Repeat the same procedure at time 6 hours.

9.3. Day 3

9.3.1. Repeat the same procedure at time 24 hours.

10. Raw Data to be Recorded

From each tank, at least at the start and end of assay, record:

water temperature

pH

salinity

From each sample and “blank”, record:

time of the sampling

OD before CTC staining

CTC fluorescence after CTC staining

OD after CTC incubation

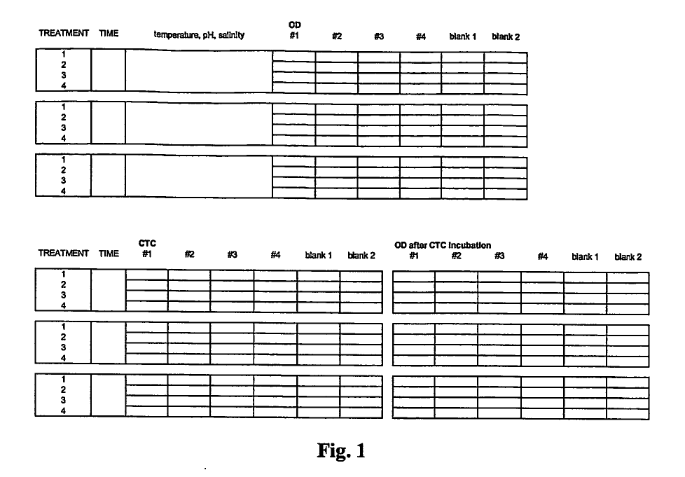

10.1. Examples of the data sheet required are given below (see FIG. 1).

11. Calculation of Results

11.1. Calculate the statistical average and standard deviation for each group of samples treatment/time.

11.2. Subtract the “blank” values from the averages for both OD and CTC. OD blanks should be near zero, and any higher value should be suspected of contamination. CTC blanks may vary but should decrease with time.

11.3. These results may be summarised in a scatter line plot to show changes in cell density and CTC fluorescence with time and treatment (see FIG. 2).

11.4. Calculate the % decrease in OD and CTC values in each treatment compared to the control (see FIG. 3).

### Example 5

### Field Application System of ECOALERT and Clean Water Test System

1. Field Application System

A field application of ECOALERT has been designed and tested. This field application consists of three main parts:

suitable format of dialysis tubes to cage the bacteria in open waters

portable turbidimeter/spectrophotometer

basket to deploy the dialysis tubes containing the bacteria into the test water.

1.2. Dialysis Tubes

Initially, the dialysis tubes used for ECOALERT were the Spectra/Por® Float-A-Lyzer® format from SPECTRUM. These tubes are 18 cm long and 10+ ml volume. They are shipped in sealed packages soaked in sodium azide preservative, which needs to be carefully rinsed with distilled water prior to use. To ensure sterility, the tubes were also UV radiated for 10 minutes. The Float-A-Lyzers have performed well during the meso-scale experiments carried out using 20-litre tanks.

However, when these dialysis tubes were used in larger flow-through systems, the format presented a few problems. When the dialysis tubes were floating inside a large tank with running water, because they are quite long and flexible, they drifted uncontrolled and easily got entangled, or sucked through the water outlet. It was also observed that the press-on-caps could leak or open accidentally during the handling process.

SPECTRUM produces another format of dialysis tubes: Spectra/Por® DispoDialyzer®. These are smaller (5 ml) tubes with screw-on cap, and they are shipped gamma irradiated and soaked in distilled water. This new format is much more appropriate for ECOALERT field applications because they are shorter and more easily handled, and the caps are well secured and leak proof Also, they are already sterile so no previous UV-irradiating is necessary.

1.3. Portable CTC Fluorometer/Turbidimeter/Spectrophotometer

A CTC portable fluorometer prototype was developed specifically for ECOALERT and is a modification of the standard handheld Aquafluor (Turner Designs), a two-channel instrument that measures chlorophyll fluorescence and turbidity.

In the CTC fluorometer prototype, the chlorophyll optical filters have been adapted according to the wavelength parameters of CTC fluorescence, with very good results. During several experiments, CTC fluorescence measurements from the portable instrument were compared to the measurements from a highly precise HPLC fluorescence detector (Jasco FP-920) resulting in a highly significant (R2=0.99) linear regression.

1.4. Deploying Basket

A preliminary device was designed for the deployment of the dialysis tubes in the field. It consists of a square basket with lid, fitted with a floating tube rack. The basket may be made of plastic or wire, of the type used to autoclave laboratory instruments (se FIG. 4a). The floating tube rack is a piece of polystyrene or foam that fits inside the basket, with a number of holes to hold the tubes by their top end (see FIG. 4b). The lid of the basket can be secured over the rack loaded with the tubes to keep them from floating off once the device is deployed in the water, and the basket must be deep enough to accommodate the length of a DispoDialyzer® tube (approximately 150 mm, see FIG. 4c).

2. Clean Water Test System as Control

Like other toxicity bioassays, ECOALERT needs to include a non-toxic control treatment so the effect from the toxicity treatments can be referred to clean, non-toxic seawater. In order to develop a clean water test system for ECOALERT, two alternative controls were considered: natural clean seawater (NSW) and artificial seawater (ASW). There is a variety of artificial salts in the market, which are commonly produced for aquariums and the aquaculture industry, and are used to make ASW from freshwater. Two brands of artificial salts were chosen based on their high quality standards: Tropical Marines Sea Salt and Crystal Sea® Marinemix. ASW was always prepared in distilled water. On the other hand, the NSW used for the ECOALERT experiments at PML was collected from a boat at the L4 oceanographic station (50° 15′N, 04° 13′W) on the continental shelf off Plymouth Sound.

Three different tests were carried out to compare the effect of two brands of artificial sea salt with NSW (Table 2). In the first test, ASW prepared with Tropical Marine was compared to NSW using two different tank sizes. In the second test, a different ASW prepared with Crystal Sea was compared to NSW. Some of that ASW was kept aerated for 4 weeks and then the same experiment was repeated in this aged ASW.

TABLE 2
Summary of the conditions during the clean water tests
using natural seawater (NSW) and two different salt
mixtures to make artificial seawater (ASW). The initial
OD of the bacterial culture is also shown.

| | | | Temper- | Tank | |
|---|---|---|---|---|---|
| | Salinity | | ature | volume | initial |
| | (%) | pH | (° C.) | (L) | OD |
| Test 1 | | | | | |
| ASW: Tropical Marine ® | 31.0 | 8.20 | 15.1 | 2, 20 | 0.041 |
| Sea Salt | | | | | |
| NSW | 31.0 | 8.13 | 15.2 | 2, 20 | 0.041 |
| Test 2 | | | | | |
| ASW: CrystalSea ® | 35.5 | 8.21 | 16.9 | 2 | 0.042 |
| Marinemix | | | | | |
| NSW | 35.5 | 8.20 | 17.7 | 2 | 0.042 |
| Test 3 | | | | | |
| ASW: CrystalSea ® | 35.5 | 8.25 | 15.9 | 2 | 0.042 |
| Marinemix, aged 4 | | | | | |
| weeks | | | | | |
| NSW | 35.5 | 8.31 | 15.8 | 2 | 0.042 |

2.1. Test 1: Tropical Marine® Sea Salt

The effect of ASW compared to NSW was different depending on the size of the tank. In the 20-litre tanks the growth of bacteria was the same for ASW and NSW, while in the 2-litre tanks the growth of bacteria in ASW was lower than the growth in NSW.

2.2. Tests 2 and 3: Crystal Sea® Marinemix

The results of ASW prepared with Crystal Sea are opposite to those from the other artificial salt: ASW enhances bacterial growth compared to NSW. However, when the test was repeated 4 weeks later using old ASW, the effect of the ASW reversed and the growth was lower than in the NSW treatment. One possible explanation is that some dissolved salts of the ASW precipitated after some time, which then altered the chemistry of the water and affected the growth of the bacteria.

2.3. Clean Water Test System

A clean water test system is necessary to validate the results of ECOALERT in the field. Results from the bacterial indicators exposed during 24 hours in a polluted site will have to be compared to the results expected in clean conditions in the same environment. One way around this problem is to use of ASW standards, prepared at the same salinity as the test water. However, the results above showed that ASW failed to recreate the conditions of NSW and can affect bacterial growth, positively or negatively. Also, the results from test 3 suggested that the chemical composition of ASW is unstable and may change with time, which again affects the bacterial indicators.

A second way to develop a clean water test is using NSW, possibly from another site considered clean that can be used as reference water. In the field, besides the ECOALERT test in a polluted site, a second test will be carried out in water from a clean area in the vicinity, or if this is not possible, in a different water system of a comparable, clean environment. Very importantly, temperature and salinity must be the same to be able to compare the results between both polluted and clean control tests.

### Example 6

### Development of New CTC Method, Clean Water Control for Field Tests, Temperature Test and Field Test System

1. New CTC Method

1.1. Background

Our objective was to optimise the new application of the redox dye 5-cyano-2,3-ditolyl tetrazolium chloride (CTC) developed in ECOALERT to estimate in vivo the respiratory activity in pure cultures of marine bacteria using fluorometric techniques. A Standard Operating Procedure has been produced, which is set out in Example 4.

The manufacturer of CTC (Polysciences) discourages the use of CTC in quantitative redox assays because it involves a lengthy additional step to dissolve the formazan necessary prior to measuring its concentration spectro-photometrically. However, in the case of bacterial cultures there should be a sufficient number of CTC-stained cells to detect the formazan fluorescence using a fluorometer.

We applied this fluorogenic CTC method to ECOALERT with excellent results. The new method was optimised to achieve a direct, precise, rapid and low-cost fluorometric application to assess the respiratory activity of cell suspensions, in particular marine bacteria cultures. The method can be easily calibrated for different bacterial strains to estimate cell numbers from fluorescence readings, and it is also able to estimate the total concentration of bacteria, and hence the proportion of actively respiring cells in sample, by simultaneously measuring the optical density of the CTC sample.

1.2. Development of New Fluorometric Application of CTC

In order to optimise the methodology, we addressed three parameters: CTC concentration, incubation time and incubation temperature.

According to the literature, the most consistent results are obtained using between 1 and 5 mM CTC as final concentration in sample. Lower concentrations yield lower numbers of CTC-stained bacteria, while higher concentrations are increasingly toxic. We found that the optimal concentration to be added to the samples was 2.5 mM CTC.

We found that the optimal incubation time was 1 hour. The level of CTC fluorescence in the samples increased rapidly during the first hour and did not change significantly in the second hour. A very similar pattern was observed in bacterial growth, estimated as optical density of the culture.

The observed results agree with other published studies and are due to the inhibitory effect of CTC on bacterial metabolism and to the CTC uptake kinetics: the intensity of CTC fluorescence per cell stabilises between 0.5-2 hours and decreases after longer incubation times because cells start releasing formazan, possibly due to cellular death. CTC kills bacteria by interrupting the respiratory chain after the bacteria take up of the CTC and the fluorescent formazan crystals are produced.

The manufacturer recommends the use of 28° C. to stain cells in suspension. However, the incubation temperatures cited in the literature are very variable. We tested the effect of incubating bacterial samples at 28° C. compared to 15° C. (standard culture growth temperature) and found no significant differences in the final fluorescence or growth. We therefore decided to use 28° C. as the standard incubation temperature for our CTC application.

All parameters that affect bacterial growth will also affect CTC reduction, so the results of the CTC method depend closely on the metabolic status of the bacteria To investigate the use of CTC as an indicator of respiratory activity in cultures of marine bacteria we applied our standardised method to a large number of case studies including laboratory tests of various chemicals and toxicity tests of natural water samples and covering a wide range of conditions of temperature, salinity, pH, concentration of nutrients in the culture medium, experimental time and concentration of cells at the start of the experiment. We found that CTC fluorescence and optical density were significantly correlated in a linear positive relationship.

To calibrate CTC fluorescence against number of respiring bacteria, duplicate samples were removed from a growing batch culture of Pseudomonas sp. during a time-course experiment. Samples were stained with CTC using the new standardised method, and the final CTC fluorescence and optical density was measured. For epifluorescence counting, 1 ml from each CTC-stained sample were preserved with 10% glutaraldehyde and kept in the fridge overnight. On the following day, the preserved sub-samples of bacteria were counter-stained with 10% DAPI (4′6-diamidino-2-phenylindole, SIGMA) for 5-10 minutes and filtered through black 0.2-μm pore membrane. The filters were then mounted onto microscopic slides and examined using an epifluorescence microscope to count number of total (DAPI-stained) and respiring (CTC-stained) bacteria.

Measurements of optical density and CTC fluorescence can be calibrated against epifluorescence counts of total DAPI-stained and respiring CTC-stained cells. Using these relationships, it is possible to estimate the proportion of actively respiring bacteria in a culture sample based on the spectro-fluorometric measurements, applying a straightforward relationship:

$$\% respiring cells = \frac{number CTC - cells}{number total DAPI - cells} \times 100 = \frac{7821 \cdot CTC}{8007 \cdot {OD}^{2} + 2309 \cdot OD} \times 100$$

where CTC is the fluorescence measured in the CTC-stained sample and OD is the optical density measured in the same sample. This equation can be recalibrated for different bacterial strains, for a rapid estimation of the proportion of actively respiring cells.

We used the equation above to estimate the proportion of respiring cells in a growing batch culture of Pseudomonas sp. using measurements of optical density and CTC fluorescence, resulting in 82.5±8.0%. The number of respiring cells was also counted using epifluorescence on samples of the same culture and the resultant proportion was 80.9±8.8%, which was very close to the fluorometric estimation.

2. Clean Water Test

In Example 5, a series of possibilities for a clean water control for ECOALERT assays were described. We investigated the use of artificial seawater mixtures, which gave contradictory results, and the use of “clean” natural seawater. In the case of field experiments, the clean control might correspond to a site or sites in the same water system (i.e. same estuary or coastal area). Alternatively, the control site may be located in a different system designated as clean so long as the conditions of the water were comparable. Both alternatives were examined during a series of toxicity tests where ECOALERT was assayed against environmental samples collected from four different estuaries: Southampton, Tamar, Falmouth and Avon (South Hams, Devon).

2.1. Single Clean Site

In the case of Southampton, we identified four sampling sites: Calshot Marshes, Fawley and Cracknore Hard, situated in potentially polluted areas inside the estuary, and Hill Head beach in the Solent, which is considered a leisure spot and therefore was used as the clean reference water. The results indicated a potential negative effect of the polluted sites compared to Hill Head. However, there was a strong gradient of salinity between Hill Head and the other three sites upstream and in consequence we cannot totally distinguish the possible effect of salinity from a potential toxic effect.

1.2. More than One Clean Site

In the case of the Tamar toxicity test, we tried a different experimental approach by grouping the collection sites and choosing more than one clean site. Three sites were identified in the Plymouth Sound area and another three sites were chosen in the Tamar Estuary area. Three clean sites were used as reference in total. In the first group of sites, water from Plymouth Sound was used as control compared to potentially polluted sites at Sutton Harbour and Devil's Point, respectively, while in the second group water collected in the vicinity of Devonport Dockyard was tested against cleaner water samples from the Lynher River.

This second experimental design was more sensitive, and of higher ecological significance, than the first one. By grouping the sites according to their common characteristics, and including one or more clean sites per group, it is possible to test their quality with better results.

2.3. Comparison Between Sites of Different Water Systems

Finally, we tested the comparison of sites from separated water systems. Water from Falmouth Estuary was tested against another water sample from the Avon in South Hams, Devon. The sampling sites were selected for having the same salinity and temperature and the two water samples were tested with ECOALERT separately.

This experimental design would be recommendable in the case when no clean site could be identified within the aquatic system of interest, or when the objective of the study is the comparison of different water systems.

3. Temperature Test

The purpose of this test was to examine the effect of temperature on the growth rate of the two bacterial strains currently used for the ECOALERT assays, Pseudomonas sp. and Vibrio natriegens. Triplicate dialysis tubes filled with log-phase cultures of each strain were separately incubated at 7, 15 and 22° C. Samples were taken at the start of the experiment and again after approximately 24 and 48 hours to measure growth and respiration activity.

The growth curve of the two strains shows a totally different adaptability to temperature. At 22° C., the shape of the growth curve was very similar in both strains, although Vibrio reached a higher final density of cells. This could have been caused by a slightly higher initial concentration of cells (Pseudomonas: 0.042 OD; Vibrio: 0.050 OD). Both strains appeared to reach stationary growth after 24 hours at 22° C. At lower temperatures, the growth curve of Vibrio flattened and slowed down but the optical density still increased during the second day of the experiment. However Pseudomonas sp. seemed to grow actively for a longer period of time at low temperatures. At the end of the experiment, the density of cells of Pseudomonas was higher at 7° C. than at 22° C., which suggests a high metabolic efficiency to low temperatures.

The changes in respiration activity, estimated as CTC fluorescence, were very similar to those observed in growth for both strains. In the case of Vibrio, the relationship between optical density and CTC fluorescence was strongly influenced by temperature and indicated that respiration activity was proportional to growth only at 22° C., while appeared distorted at lower temperatures. By contrast, Pseudomonas showed an exponential positive relationship between CTC fluorescence and optical density that was independent of temperature.

These results suggest that Pseudomonas sp. is better adapted to growing in cold waters than Vibrio natriegens. This fact makes Pseudomonas a highly suitable bacterial strain to be used for ECOALERT assays, particularly in the field where the temperatures are below 15° C. for most of the year.

4. Field Test System

The prototype deployment device that was described in Example 5 was tested during laboratory experiments of natural water samples and also during real field deployments, with good results. Three tests were carried out at Plymouth Marine Laboratory using water samples collected from different sites: Southampton Water, Falmouth Estuary, Avon Estuary, and Tamar Estuary. In every test, the sensitivity of ECOALERT was examined by comparing the results from polluted sites against clean sites.

4.1. Laboratory ECOALERT Assays of Natural Water Samples in the Laboratory

All the tests were run following the same routine. In the field, water samples were collected in clean 5-litre carboys and brought back to the laboratory. The conditions of temperature, salinity and pH of the water on the site were measured whenever possible.

Back at the laboratory, the carboys were kept in the dark and refrigerated at 7° C. until used (within 3 days). The day before the experiment, the water in the carboys was used to fill clean 2-litre HDPE, wide mouth experimental bottles. The bottles were left acclimating overnight in a chamber set at approximately the in situ temperature, gently stirred with magnetic bars before the test started. At the same time, a pre-culture of bacteria was prepared according to the Standard Procedure set out in Example 4, and was incubated at 15° C. during the night.

In the morning of the experiment day, an exponentially growing suspension of bacteria was prepared from the pre-culture. Once the suspension reached the desired concentration of cells as measured by optical density, it was filled into the dialysis tubes. Four dialysis tubes were put floating inside each bottle: three tubes containing bacterial culture and one “blank” tube containing medium-only without cells.

The growth and activity of the bacteria in each “site” treatment was measured during a time-course experiment at approximately 1, 6, 24 and 48 hours. Samples of 1.9 ml were removed from each dialysis tube with a sterile Pasteur pipette into fluorometer polystyrene cuvettes. The optical density was measured in the spectrophotometer at A540 nm wavelength. Immediately after, the samples in the cuvettes were stained with 2.5 mM final concentration of the redox dye CTC and incubated at 28° C. in the dark for 1 h. At the end of the incubation, fluorescence from the CTC-stained cells was measured in the fluorometer at 488 nm excitation, 630 nm emission wavelengths to estimate the respiratory activity of the bacteria The final optical density was also measured.

4.1.1. ECOALERT Assay of Samples from Southampton Water System

Water was collected from the shore along the Southampton channel on 31 Jan. 2003. One sample was taken from Hill Head beach to be used as the clean site reference, and three more samples were taken from potentially polluted sites: Calshot Marshes, Fawley and Cracknore Hard.

The ECOALERT test was carried out twice, each time using a different bacteria species: Pseudomonas sp. and Vibrio natriegens. The growth and respiration activity of the bacteria were measured after 2, 6 and 24 hours exposure.

The conditions of the water in the experimental 2-litre bottles measured during the course of this assay are summarised in Table 3.

TABLE 3
Conditions of the water in the experimental bottles
during the ECOALERT toxicity tests of Southampton
Water, using two different bacterial cultures.

| | Pseudomonas | | | Vibrio natriegens | | |
|---|---|---|---|---|---|---|
| | test | | | test | | |
| | Temp. | | Salinity | Temp. | | Salinity |
| | (° C.) | pH | (%) | (° C.) | pH | (%) |
| Cracknore Hard | 17.1 | 8.05 | 23.0 | 20.4 | 8.03 | 22.0 |
| Fawley | 17.4 | 7.96 | 31.5 | 20.5 | 7.93 | 30.0 |
| Calshot Marshes | 17.2 | 7.96 | 30.0 | 20.1 | 7.92 | 29.5 |
| Hill Head | 17.9 | 7.97 | 33.5 | 20.0 | 7.90 | 32.0 |

A strong salinity gradient can be appreciated between Cracknore Hard and Hill Head. The ECOALERT assay showed that growth and also respiratory activity of Pseudomonas decreased after 24 hours in the Cracknore water compared to Hill Head. A similar trend was also observed in Vibrio after 6 hours.

To measure the magnitude of the effect of the polluted samples, the % change was calculated referred to the control site as. After 24 hours exposure to Cracknore Hard water, the growth of Pseudomonas reduced by 13%, which indicates a negative effect on growth. In the case of Vibrio, the optical density decreased by 7-13% in all treatments compared to the clean site. The respiration activity, as indicated by CTC fluorescence, also decreased in the same magnitude for both bacteria.

The salinity gradient across the water samples may have affected the outcome of the experiment, so that the final effect on growth in the polluted water samples compared to the control is a combination of toxicity and the effect of the difference in salinity. For example, in the case of the Cracknore Hard water, salinity was 23.0% compared to 33.5% of Hill Head water (Table 3). Using the results from a test on the effect of salinity on Pseudomonas sp. that was carried out during Example 2 it is possible to predict the change on bacterial growth due to the salinity effect alone. This change, for a 24 h exposure, would represent a 6% decrease of bacterial growth at 23.0% salinity compared to the growth at 33.5% salinity. This means that in the 13.2% growth reduction observed in the Cracknore water treatment, there is still at least a potential ˜7% decrease that can be attributed to toxicity.

4.1.2. ECOALERT Assay of Samples from Plymouth Sound and Tamar Estuary

Water was collected on board the RV Tamaris from six sites along the Tamar Estuary on 17 Mar. 2003. Plymouth Sound was used as a clean site and compared to Sutton Harbour and Devil's Point sites, while St German's and the Lynher mouth were used as clean reference sites for a water sample collected in the proximity of Devonport Royal Dockyard. The surface water temperature was 11.0-11.5° C. and the salinity (%) gradient was as follows: Plymouth Sound, 35.5; Devil's Point, 33.0; Sutton Harbour, 32.0; Devonport and Lynher mouth, 24.0; St German's, 23.0. Growth and respiration activity of Pseudomonas sp. exposed to water from each site were measured after 1, 7, 24 and 49 hours. In general, ECOALERT showed that in most of the polluted sites growth and respiration activity of Pseudomonas increased compared to the controls. For example, the respiration activity indicator was significantly higher in the Devonport water compared to the average value from the clean sites St German's and the Lynher mouth. Similarly, respiration was also significantly higher in Devil's Point compared to Plymouth Sound. In the Sutton Harbour water, bacteria appeared to slow down growth after 24 hours. The optical density indicator showed an identical pattern.

The results indicated a general 15-40% increase in bacterial respiration after 1-2 days exposure to water from the polluted sites, compared to the controls.

The exception was the case of the Sutton Harbour water, which appeared to cause 18% reduction in the respiration activity of bacteria after 48 hours. The same pattern was observed in the optical density, indicating that bacterial growth slowed down after 24 hours as it entered stationary phase.

These results suggest that there are potential sources of eutrophication in these study sites, which is reflected in enhanced bacterial activity and population growth.

4.1.3. ECOALERT Assay of Samples from Falmouth and Avon Estuaries

Water was collected from the shore from Restronguet Creek in the Falmouth Estuary on 10 Mar. 2003, and in the Avon Estuary from the tidal end of the river before Aveton Grifford on 23 Mar. 2003. The temperature of the surface water was approximately 11.5° C. and the salinity was 15% in both occasions. The two water types were separately tested with ECOALERT in duplicate 2-litre bottles and the results were compared considering the Avon water as the clean site reference for the Falmouth water. Each test lasted for 2 days and samples were taken at 1, 7, 25 and 52 hours to measure bacterial growth and respiration. The experimental bottles were kept at in situ temperature (11.5° C.) and the water pH measured during the test was 7.64 (Falmouth) and 7.80 (Avon).

No significant differences were found in growth or respiration activity between the bacteria exposed to Falmouth or Avon water. This conclusion is very interesting as we expected to see some negative effect in the Falmouth water. One possible explanation is that, because the water at Falmouth was collected at high tide, the incoming water from the sea might have diluted down the levels of pollutants. A second explanation is that the concentrations of pollutants (i.e. heavy metals) that are present in Restronguet Creek are such that there is a hormetic (positive) response by the bacteria. To test these hypotheses, further tests would be necessary.

4.2. Deployment in Controlled Flow-Through System

As a preliminary approach to the field, a test was carried out in the flow through system facilities at Rogaland Research in Stavanger, Norway, from 31 Mar. to 4 Apr. 2003, using the prototype of deployment device that was developed during the Example 3. The test was coordinated within a workshop where the effect of four chemicals was being tested on a suite of bio-indicators from four animal species: mussel, cod, turbot, and spider crab.

This experiment was considered as a trial deployment for ECOALERT and proved to be most useful in that it became necessary to improvise some changes due to unforeseen problems found during the process of preparing the dialysis tubes in the floating racks and filling them with the culture. The dialysis tubes used for the field device of ECOALERT (DispoDialyzer®, Spectrum) are supplied soaked in distilled water inside a plastic case. That water must be emptied and the tubes rinsed with more distilled water. The tubes may be filled with cultures before or after placing them in the rack, and all these preparations must be done very gently to avoid tearing the membrane.

When the dialysis tubes are left air drying for more than a few minutes the membrane rapidly deteriorates, shrinks and stiffens, and it becomes opaque, brittle and very difficult to handle, although it will slowly recover when moistened. To avoid this happening, the tubes were refilled with the bacterial suspension immediately after unpacking and removing the distilled water inside.

For better results and easier handling, each dialysis tube should be sampled only once. Sampling the content of the same tube repeatedly increases the risk of contamination. It also increases the total handling time because it is necessary to condition the tube after sampling to remove the air space created. This means that a sufficient number of tubes must be anticipated for each test so that enough number of replicates and time-points can be processed.

The experimental setup involved 16 tanks (4 animal species 4 exposure treatments) connected in a flow-through system with a direct intake of seawater from 80 m depth in the Stavanger fjord, outside the laboratory. For each one of the test animals, three tanks were connected to a chemostate system which maintained a constant concentration of each of the following chemicals: 50 ppb bisphenol A (BPH), 50 ppb diallyl phthalate (DAP), and 5 ppb tetrabromodiphenylether or PBDE-47 (TBDE). The forth tank had no chemical supply and was used as control. ECOALERT was deployed in the mussel tanks, one basket per tank. The water conditions in all the tanks were: 7.5° C. temperature, pH 7.78 and 35% salinity.

Each deploying basket of ECOALERT was equipped with 3 dialysis tubes with Pseudomonas, 3 tubes filled with Vibrio natriegens and 3 dialysis tubes with medium-only without cells. Each dialysis tube was repeatedly sampled at 1, 24, 48 and 72 hours. Due to the low temperature of the water (7.5° C.) a low bacterial growth rate was expected and because of that the duration of the experiment was expanded to 72 hours. At every time-point, growth and respiration activity of the bacteria were measured as turbidity and CTC fluorescence respectively using the handheld CTC fluorometer/turbidimeter. This handheld instrument was calibrated with a desktop fluorometer and desktop spectrophotometer as set out in Example 5.

The overnight pre-culture and the log-phase culture of each bacterial strain were prepared in the same fashion as has been described for the toxicity tests carried out in the laboratory.

The unexpected problems encountered during the preparation of the dialysis tubes may have affected the outcome of this ECOALERT test. For example, the density of bacteria measured after the first hour was significantly higher in the Vibrio tubes (13.5±1.3 turbidity units) than in the Pseudomonas tubes (5.2±0.6 turbidity units). This may have been caused involuntarily by the delay in filling up the tubes at the beginning of the experiment; as the Pseudomonas tubes were being filled the membrane of the ones reserved for Vibrio had started to dry out. One possible explanation is that the permeability of the membrane was affected and the dialysis was less effective than in the Pseudomonas tubes. As a consequence, the medium nutrients contained inside the tube may have been retained, at least temporarily, stimulating the growth of Vibrio.

After the first 24 hours, the growth rate of Vibrio clearly decreased compared to Pseudomonas, which, slowly but steadily grew in number and respiratory activity during the three days of the experiment. This result was very interesting and confirmed the adverse effect of low temperature on the growth of Vibrio previously reported in the laboratory.

Bacterial respiration measured in the Pseudomonas tubes indicated a clear negative effect of BPH on the bacteria after 72 hours, and a possible positive effect of TBDE, although in this case the difference was not statistically significant.

No clear results could be obtained from the test using with Vibrio due to the adverse effect that the low temperature had on their growth rate. In consequence, the Vibrio test was discarded and the results were not analysed for a toxicity effect. Even if the results of the toxicity assay itself were partly inconclusive, as a whole this experiment at Rogaland was of key importance in developing and improving the protocol of field deployment of ECOALERT. Of wider significance, however, is the observation that BPH, which is a known endocrine disruptor in higher animals, also had an impact on bacteria.

1. A method for the determination of water quality, which method comprises:
contacting a vessel comprising a population of bacteria with a water sample to be tested, said vessel comprising a semi-permeable material which allows the water sample to pass therethrough and contact said bacteria; and
determining the growth rate of the bacteria and proportion of respiring bacteria in the vessel,
thereby to determine the water quality of the water sample.

2. A method according to claim 1, wherein a test sample is withdrawn from the vessel and the growth rate of the bacteria and proportion of respiring bacteria in the test sample is determined.

3. A method according to claim 1, wherein two or more vessels, each comprising a population of bacteria, are simultaneously contacted with the water sample to be tested.

4. A method according to claim 1, wherein the semi-permeable material is dialysis tubing.

5. A method according to claim 1, wherein the semi-permeable material has a molecular weight cut-off of from about 100 Da to about 300 kDa.

6. A method according to claim 5, wherein the semi-permeable material has a molecular weight cut-off of about 60 kDa.

7. A method according to claim 5, wherein the semi-permeable material has a molecular weight cut-off of about 25 kDa.

8. A method according to claim 1, wherein the population of bacteria comprises one or more species of marine or freshwater bacteria or a mixture of any thereof.

9. A method according to claim 1, wherein the population of bacteria comprises genetically modified bacteria.

10. A method according to claim 8, wherein the population of bacteria comprises one or more Pseudomonas and/or Vibrio species.

11. A method according to claim 1, wherein the population of bacteria is in the exponential growth phase when the vessel is contacted with the water sample to be tested.

12. A method according to claim 2, wherein more than one test sample is withdrawn from the or each vessel, each test sample being withdrawn at a different predetermined time point.

13. A method according to claim 12, wherein a first sample is withdrawn within ½ h of the contacting step, a second sample is withdrawn at about 6 h after the contacting step and a third sample is withdrawn at about 24 h after the contacting step.

14. A method according to claim 12, wherein a first sample is withdrawn within ½ h of the contacting step and a further sample is withdrawn at each hour after the contacting step up to 24 h after the contacting step.

15. A method according to claim 1, wherein the growth rate of the bacteria in the vessel or test sample is determined by determining the optical density and/or the turbidity of the bacterial population in the vessel or test sample.

16. A method according to claim 15, wherein the optical density is determined at a wavelength of about 540 nm.

17. A method according to claim 1, wherein the proportion of respiring bacteria in the vessel or test sample is determined by contacting the bacterial population in the vessel or test sample with a redox dye and determining the level of fluorescence of the resulting bacterial population in the vessel or test sample.

18. A method according to claim 17, wherein the level of fluorescence of the resulting bacterial population in the vessel or test sample is determined by exciting the bacterial population in the vessel or test sample using light of a predetermined intensity at a first predetermined wavelength and measuring the intensity of fluorescence at a second predetermined wavelength.

19. A method according to claim 17, wherein the redox dye is 5-cyano-2,3-ditolyl tetrazolium chloride.

20. A method according to claim 1, wherein a decrease in bacterial growth rate and/or in the proportion of respiring bacteria in the vessel of test sample as compared to the corresponding bacterial growth rate and/or proportion of respiring bacterial in a control water sample is indicative of a decrease in water quality.

21. A method according to claim 1 which is adapted to be carried out in the field.

22. A detection device for use in a method according to claim 1 which device is arranged to determine the optical density and/or turbidity of a bacterial culture and also to determine the level of fluorescence of a bacterial culture contacted with a redox dye.

23. A detection device according to claim 22, said device comprising means for determining the optical density of a bacterial culture at a wavelength of about 540 nm.

24. A detection device according to claim 22, comprising a photodiode detector for determining the optical density of a bacterial culture.

25. A detection device according to claim 22, said device comprising means for determining the level of fluorescence of a bacterial culture contacted with a redox dye by exciting the culture using light of a predetermined intensity at a first predetermined wavelength and measuring the intensity of fluorescence at a second predetermined wavelength.

26. A detection device according to claim 25, wherein said means for determining the level of fluorescence of a bacterial culture contacted with a redox dye comprises a light source for emitting said light of said first predetermined wavelength and a detector for detecting light of said second predetermined wavelength.

27. A detection device according to claim 25, wherein the first predetermined wavelength is about 488 nm and the second predetermined wavelength is about 630 nm.

28. A detection device according to claim 22, which is a hand-held device.

29. A kit of components for use in carrying out a method according to claim 1, which kit of components comprises:
one or more vessels comprising a semi-permeable material which allows a water sample to pass therethrough;
a non-watertight carrying structure to carry the one or more vessels; and
a member for securing the position of the one or more vessels within the carrying structure and/or for providing flotation of the carrying structure.

30. A kit of components according to claim 29, wherein said member is a buoyant member for providing flotation of the whole kit of components.

31. A kit of components according to claim 29, wherein the one or more vessels are each dialysis tubes.

32. A kit of components according to claim 29 which further comprises a detection device which device is arranged to determine the optical density and/or turbidity of a bacterial culture and also to determine the level of fluorescence of a bacterial culture contacted with a redox dye.

33. A kit of components according to claim 29 which comprises means for transporting together the other components of the kit.

34. A kit of components according to claim 29, wherein said non-watertight carrying structure is a basket.

35. A deployment device suitable for carrying one or more vessels that each comprise a semi-permeable material which allows a water sample to pass into said vessels, said deployment device comprising a non-watertight housing and means for securing the one or more vessels within the housing and/or means for providing flotation of the deployment device.

36. A sampling device for use in a method according to claim 1, which sampling device comprises:
(i) a sampling chamber which is adapted to accept one or more vessels comprising a semi-permeable material which allows a water sample to pass therethrough; and
(ii) a closure member selectively switchable between two or more positions, wherein:
in one of the said positions the closure member seals the sampling chamber to prevent ingress of water into the sampling chamber when the device is submerged in water; and
in a second of the said positions the closure member is open to allow ingress of water into the sampling chamber when the device is submerged in water.

37. A sampling device according to claim 36, which comprises two or more sampling chambers, each with a closure member, wherein the sampling chambers are arranged so that each sampling chamber is positioned at a different depth when the sampling device is submerged in water.

38. A sampling device according to claim 36, wherein said sampling chambers are connected together by a cable so that said sampling device may be deployed by lowering said cable into water to position said two or more sampling chambers at respectively different depths.

39. A sampling device according to claim 38, wherein said closure member is activated by electro-mechanical means.

40. A sampling device according to claim 39, wherein said cable is arranged to carry actuation signals to open or close said closure member.

41. A sampling device according to claim 39, wherein said closure member comprises a plate having one or more water ingress holes therethrough and one or more index holes therethrough, said plate being moveable so as to selectively open the sampling chamber in the second position by presenting a water ingress hole over said sampling chamber.

42. A sampling device according to claim 41, wherein said plate is spring-loaded and is indexed by a solenoid actuator cooperating with said one or more index holes.

43. A sampling device according to claim 41, further comprising a microswitch for providing feedback as to the open or closed state of said plate.
